SEQUENCE LISTING (50 sequences)

SEQ ID NO: 1 - DNA, 3682 bp, Homo sapiens
gcgagcgcagcggagcctggagagaaggcgctgggctgcgagggcgcgagggcgcgagggcagggggcaaccggaccccgcccgcacccatggcgcccgtcgccgtctgggccgcgctggccgtcggactggagctctgggctgcggcgcacgccttgcccgcccaggtggcatttacaccctacgccccggagcccgggagcacatgccggctcagagaatactatgaccagacagctcagatgtgctgcagcaaatgctcgccgggccaacatgcaaaagtcttctgtaccaagacctcggacaccgtgtgtgactcctgtgaggacagcacatacacccagctctggaactgggttcccgagtgcttgagctgtggctcccgctgtagctctgaccaggtggaaactcaagcctgcactcgggaacagaaccgcatctgcacctgcaggcccggctggtactgcgcgctgagcaagcaggaggggtgccggctgtgcgcgccgctgcgcaagtgccgcccgggcttcggcgtggccagaccaggaactgaaacatcagacgtggtgtgcaagccctgtgccccggggacgttctccaacacgacttcatccacggatatttgcaggccccaccagatctgtaacgtggtggccatccctgggaatgcaagcatggatgcagtctgcacgtccacgtcccccacccggagtatggccccaggggcagtacacttaccccagccagtgtccacacgatcccaacacacgcagccaactccagaacccagcactgctccaagcacctccttcctgctcccaatgggccccagccccccagctgaagggagcactggcgacttcgctcttccagttggactgattgtgggtgtgacagccttgggtctactaataataggagtggtgaactgtgtcatcatgacccaggtgaaaaagaagcccttgtgcctgcagagagaagccaaggtgcctcacttgcctgccgataaggcccggggtacacagggccccgagcagcagcacctgctgatcacagcgccgagctccagcagcagctccctggagagctcggccagtgcgttggacagaagggcgcccactcggaaccagccacaggcaccaggcgtggaggccagtggggccggggaggcccgggccagcaccgggagctcagattcttcccctggtggccatgggacccaggtcaatgtcacctgcatcgtgaacgtctgtagcagctctgaccacagctcacagtgctcctcccaagccagctccacaatgggagacacagattccagcccctcggagtccccgaaggacgagcaggtccccttctccaaggaggaatgtgcctttcggtcacagctggagacgccagagaccctgctggggagcaccgaagagaagcccctgccccttggagtgcctgatgctgggatgaagcccagttaaccaggccggtgtgggctgtgtcgtagccaaggtgggctgagccctggcaggatgaccctgcgaaggggccctggtccttccaggcccccaccactaggactctgaggctctttctgggccaagttcctctagtgccctccacagccgcagcctccctctgacctgcaggccaagagcagaggcagcgagttgtggaaagcctctgctgccatggcgtgtccctctcggaaggctggctgggcatggacgttcggggcatgctggggcaagtccctgactctctgtgacctgccccgcccagctgcacctgccagcctggcttctggagcccttgggttttttgtttgtttgtttgtttgtttgtttgtttctccccctgggctctgccccagctctggcttccagaaaaccccagcatccttttctgcagaggggctttctggagaggagggatgctgcctgagtcacccatgaagacaggacagtgcttcagcctgaggctgagactgcgggatggtcctggggctctgtgcagggaggaggtggcagccctgtagggaacggggtccttcaagttagctcaggaggcttggaaagcatcacctcaggccaggtgcagtggctcacgcctatgatcccagcactttgggaggctgaggcgggtggatcacctgaggttaggagttcgagaccagcctggccaacatggtaaaaccccatctctactaaaaatacagaaattagccgggcgtggtggcgggcacctatagtcccagctactcagaagcctgaggctgggaaatcgtttgaacccgggaagcggaggttgcagggagccgagatcacgccactgcactccagcctgggcgacagagcgagagtctgtctcaaaagaaaaaaaaaagcaccgcctccaaatgccaacttgtccttttgtaccatggtgtgaaagtcagatgcccagagggcccaggcaggccaccatattcagtgctgtggcctgggcaagataacgcacttctaactagaaatctgccaattttttaaaaaagtaagtaccactcaggccaacaagccaacgacaaagccaaactctgccagccacatccaaccccccacctgccatttgcaccctccgccttcactccggtgtgcctgcagccccgcgcctccttccttgctgtcctaggccacaccatctcctttcagggaatttcaggaactagagatgactgagtcctcgtagccatctctctactcctacctcagcctagaccctcctcctcccccagaggggtgggttcctcttccccactccccaccttcaattcctgggccccaaacgggctgccctgccactttggtacatggccagtgtgatcccaagtgccagtcttgtgtctgcgtctgtgttgcgtgtcgtgggtgtgtgtagccaaggtcggtaagttgaatggcctgccttgaagccactgaagctgggattcctccccattagagtcagccttccccctcccagggccagggccctgcagaggggaaaccagtgtagccttgcccggattctgggaggaagcaggttgaggggctcctggaaaggctcagtctcaggagcatggggataaaggagaaggcatgaaattgtctagcagagcaggggcagggtgataaattgttgataaattccactggacttgagcttggcagctgaactattggagggtgggagagcccagccattaccatggagacaagaagggttttccaccctggaatcaagatgtcagactggctggctgcagtgacgtgcacctgtactcaggaggctgaggggaggatcactggagcccaggagtttgaggctgcagcgagctatgatcgcgccactacactccagcctgagcaacagagtgagaccctgtctcttaaagaaaaaaaaagtcagactgctgggactggccaggtttctgcccacattggacccacatgaggacatgatggagcgcacctgccccctggtggacagtcctgggagaacctcaggcttccttggcatcacagggcagagccgggaagcgatgaatttggagactctgtggggccttggttcccttgtgtgtgtgtgttgatcccaagacaatgaaagtttgcactgtatgctggacggcattcctgcttatcaataaacctgtttgttttaaaaaaaa

SEQ ID NO: 2 - protein, 461 aa, Homo sapiens
MAPVAVWAALAVGLELWAAAHALPAQVAFTPYAPEPGSTCRLREYYDQTAQMCCSKCSPGQHAKVFCTKTSDTVCDSCEDSTYTQLWNWVPECLSCGSRCSSDQVETQACTREQNRICTCRPGWYCALSKQEGCRLCAPLRKCRPGFGVARPGTETSDVVCKPCAPGTFSNTTSSTDICRPHQICNVVAIPGNASMDAVCTSTSPTRSMAPGAVHLPQPVSTRSQHTQPTPEPSTAPSTSFLLPMGPSPPAEGSTGDFALPVGLIVGVTALGLLIIGVVNCVIMTQVKKKPLCLQREAKVPHLPADKARGTQGPEQQHLLITAPSSSSSSLESSASALDRRAPTRNQPQAPGVEASGAGEARASTGSSDSSPGGHGTQVNVTCIVNVCSSSDHSSQCSSQASSTMGDTDSSPSESPKDEQVPFSKEECAFRSQLETPETLLGSTEEKPLPLGVPDAGMKPS

SEQ ID NO: 3 - DNA, 3417 bp, Homo sapiens
cggaggcagcgagaaagcgcagccaggcggctgctcggcgttctctcaggtgactgctcggagttctcccagtgtttggtgttgcaagcaggatccaaaggagacctatagtgactcccaggagctcttagtgaccaagtgaaggtacctgtggggctcattgtgcccattgctctttcactgctttcaactggtagttgtgggttgaagcactggacaatgccacatactttgtggatggtgtgggtcttgggggtcatcatcagcctctccaaggaagaatcctccaatcaggcttctctgtcttgtgaccgcaatggtatctgcaagggcagctcaggatctttaaactccattccctcagggctcacagaagctgtaaaaagccttgacctgtccaacaacaggatcacctacattagcaacagtgacctacagaggtgtgtgaacctccaggctctggtgctgacatccaatggaattaacacaatagaggaagattctttttcttccctgggcagtcttgaacatttagacttatcctataattacttatctaatttatcgtcttcctggttcaagcccctttcttctttaacattcttaaacttactgggaaatccttacaaaaccctaggggaaacatctcttttttctcatctcacaaaattgcaaatcctgagagtgggaaatatggacaccttcactaagattcaaagaaaagattttgctggacttaccttccttgaggaacttgagattgatgcttcagatctacagagctatgagccaaaaagtttgaagtcaattcagaatgtaagtcatctgatccttcatatgaagcagcatattttactgctggagatttttgtagatgttacaagttccgtggaatgtttggaactgcgagatactgatttggacactttccatttttcagaactatccactggtgaaacaaattcattgattaaaaagtttacatttagaaatgtgaaaatcaccgatgaaagtttgtttcaggttatgaaacttttgaatcagatttctggattgttagaattagagtttgatgactgtacccttaatggagttggtaattttagagcatctgataatgacagagttatagatccaggtaaagtggaaacgttaacaatccggaggctgcatattccaaggttttacttattttatgatctgagcactttatattcacttacagaaagagttaaaagaatcacagtagaaaacagtaaagtttttctggttccttgtttactttcacaacatttaaaatcattagaatacttggatctcagtgaaaatttgatggttgaagaatacttgaaaaattcagcctgtgaggatgcctggccctctctacaaactttaattttaaggcaaaatcatttggcatcattggaaaaaaccggagagactttgctcactctgaaaaacttgactaacattgatatcagtaagaatagttttcattctatgcctgaaacttgtcagtggccagaaaagatgaaatatttgaacttatccagcacacgaatacacagtgtaacaggctgcattcccaagacactggaaattttagatgttagcaacaacaatctcaatttattttctttgaatttgccgcaactcaaagaactttatatttccagaaataagttgatgactctaccagatgcctccctcttacccatgttactagtattgaaaatcagtaggaatgcaataactacgttttctaaggagcaacttgactcatttcacacactgaagactttggaagctggtggcaataacttcatttgctcctgtgaattcctctccttcactcaggagcagcaagcactggccaaagtcttgattgattggccagcaaattacctgtgtgactctccatcccatgtgcgtggccagcaggttcaggatgtccgcctctcggtgtcggaatgtcacaggacagcactggtgtctggcatgtgctgtgctctgttcctgctgatcctgctcacgggggtcctgtgccaccgtttccatggcctgtggtatatgaaaatgatgtgggcctggctccaggccaaaaggaagcccaggaaagctcccagcaggaacatctgctatgatgcatttgtttcttacagtgagcgggatgcctactgggtggagaaccttatggtccaggagctggagaacttcaatccccccttcaagttgtgtcttcataagcgggacttcattcctggcaagtggatcattgacaatatcattgactccattgaaaagagccacaaaactgtctttgtgctttctgaaaactttgtgaagagtgagtggtgcaagtatgaactggacttctcccatttccgtctttttgatgagaacaatgatgctgccattctcattcttctggagcccattgagaaaaaagccattccccagcgcttctgcaagctgcggaagataatgaacaccaagacctacctggagtggcccatggacgaggctcagcgggaaggattttgggtaaatctgagagctgcgataaagtcctaggttcccatatttaagaccagtctttgtctagttgggatctttatgtcactagttatagttaagttcattcagacataattatataaaaactacgtggatgtaccgtcatttgaggacttgcttactaaaactacaaaacttcaaattttgtctggggtgctgttttataaacatatgccagatttaaaaattggtttttggtttttcttttttctatgagataaccatgatcataagtctattactgatatctgaatatagtcccttggtatccaagggaattggttgcaggatcctcgtggatatcaaaattcatagatgatcaagtcccttataagagtggcatagtatttgcatataacctgtgtacattctcctgtatactttaaatcatctctagattacttatgatacccaatacaatgtaaatactatgtaaatagttgtactgtctttttatttatattattattgttattttttattttcaaaatttttaaaacatacttttgatccacagttggttgacttcatggatgcagaacccatggatatagagggccaactgtaatctgtagcaactggcttagttcattaggaaacagcacaaatgaacttaagattctcaatgactgtgtcattctttcttcctgctaagagactcctctgtggccacaaaaggcattctctgtcctacctagctgtcacttctctgtgcagctgatctcaagagcaacaaggcaaagtatttggggcactccccaaaacttgttgctattcctagaaaaaagtgctgtgtatttcctattaaactttacaggatgagaaaaaaaaaaaaaaaaa

SEQ ID NO: 4 - protein, 784 aa, Homo sapiens
MPHTLWMVWVLGVIISLSKEESSNQASLSCDRNGICKGSSGSLNSIPSGLTEAVKSLDLSNNRITYISNSDLQRCVNLQALVLTSNGINTIEEDSFSSLGSLEHLDLSYNYLSNLSSSWFKPLSSLTFLNLLGNPYKTLGETSLFSHLTKLQILRVGNMDTFTKIQRKDFAGLTFLEELEIDASDLQSYEPKSLKSIQNVSHLILHMKQHILLLEIFVDVTSSVECLELRDTDLDTFHFSELSTGETNSLIKKFTFRNVKITDESLFQVMKLLNQISGLLELEFDDCTLNGVGNFRASDNDRVIDPGKVETLTIRRLHIPRFYLFYDLSTLYSLTERVKRITVENSKVFLVPCLLSQHLKSLEYLDLSENLMVEEYLKNSACEDAWPSLQTLILRQNHLASLEKTGETLLTLKNLTNIDISKNSFHSMPETCQWPEKMKYLNLSSTRIHSVTGCIPKTLEILDVSNNNLNLFSLNLPQLKELYISRNKLMTLPDASLLPMLLVLKISRNAITTFSKEQLDSFHTLKTLEAGGNNFICSCEFLSFTQEQQALAKVLIDWPANYLCDSPSHVRGQQVQDVRLSVSECHRTALVSGMCCALFLLILLTGVLCHRFHGLWYMKMMWAWLQAKRKPRKAPSRNICYDAFVSYSERDAYWVENLMVQELENFNPPFKLCLHKRDFIPGKWIIDNIIDSIEKSHKTVFVLSENFVKSEWCKYELDFSHFRLFDENNDAAILILLEPIEKKAIPQRFCKLRKIMNTKTYLEWPMDEAQREGFWVNLRAAIKS

SEQ ID NO: 5 - DNA, 2672 bp, Homo sapiens
cttccagagagcaatatggctggttccccaacatgcctcaccctcatctatatcctttggcagctcacagggtcagcagcctctggacccgtgaaagagctggtcggttccgttggtggggccgtgactttccccctgaagtccaaagtaaagcaagttgactctattgtctggaccttcaacacaacccctcttgtcaccatacagccagaagggggcactatcatagtgacccaaaatcgtaatagggagagagtagacttcccagatggaggctactccctgaagctcagcaaactgaagaagaatgactcagggatctactatgtggggatatacagctcatcactccagcagccctccacccaggagtacgtgctgcatgtctacgagcacctgtcaaagcctaaagtcaccatgggtctgcagagcaataagaatggcacctgtgtgaccaatctgacatgctgcatggaacatggggaagaggatgtgatttatacctggaaggccctggggcaagcagccaatgagtcccataatgggtccatcctccccatctcctggagatggggagaaagtgatatgaccttcatctgcgttgccaggaaccctgtcagcagaaacttctcaagccccatccttgccaggaagctctgtgaaggtgctgctgatgacccagattcctccatggtcctcctgtgtctcctgttggtgcccctcctgctcagtctctttgtactggggctatttctttggtttctgaagagagagagacaagaagagtacattgaagagaagaagagagtggacatttgtcgggaaactcctaacatatgcccccattctggagagaacacagagtacgacacaatccctcacactaatagaacaatcctaaaggaagatccagcaaatacggtttactccactgtggaaataccgaaaaagatggaaaatccccactcactgctcacgatgccagacacaccaaggctatttgcctatgagaatgttatctagacagcagtgcactcccctaagtctctgctcaaaaaaaaaacaattctcggcccaaagaaaacaatcagaagaattcactgatttgactagaaacatcaaggaagaatgaagaacgttgacttttttccaggataaattatctctgatgcttctttagatttaagagttcataattccatccactgctgagaaatctcctcaaacccagaaggtttaatcacttcatcccaaaaatgggattgtgaatgtcagcaaaccataaaaaaagtgcttagaagtattcctatagaaatgtaaatgcaaggtcacacatattaatgacagcctgttgtattaatgatggctccaggtcagtgtctggagtttcattccatcccagggcttggatgtaaggattataccaagagtcttgctaccaggagggcaagaagaccaaaacagacagacaagtccagcagaagcagatgcacctgacaaaaatggatgtattaattggctctataaactatgtgcccagcactatgctgagcttacactaattggtcagacgtgctgtctgccctcatgaaattggctccaaatgaatgaactactttcatgagcagttgtagcaggcctgaccacagattcccagagggccaggtgtggatccacaggacttgaaggtcaaagttcacaaagatgaagaatcagggtagctgaccatgtttggcagatactataatggagacacagaagtgtgcatggcccaaggacaaggacctccagccaggcttcatttatgcacttgtgctgcaaaagaaaagtctaggttttaaggctgtgccagaacccatcccaataaagagaccgagtctgaagtcacattgtaaatctagtgtaggagacttggagtcaggcagtgagactggtggggcacggggggcagtgggtacttgtaaacctttaaagatggttaattcattcaatagatatttattaagaacctatgcggcccggcatggtggctcacacctgtaatcccagcactttgggaggccaaggtgggtgggtcatctgaggtcaggagttcaagaccagcctggccaacatggtgaaaccccatctctactaaagatacaaaaatttgctgagcgtggtggtgtgcacctgtaatcccagctactcgagaggccaaggcatgagaatcgcttgaacctgggaggtggaggttgcagtgagctgagatggcaccactgcactccggcctaggcaacgagagcaaaactccaatacaaacaaacaaacaaacacctgtgctaggtcagtctggcacgtaagatgaacatccctaccaacacagagctcaccatctcttatacttaagtgaaaaacatggggaaggggaaaggggaatggctgcttttgatatgttccctgacacatatcttgaatggagacctccctaccaagtgatgaaagtgttgaaaaacttaataacaaatgcttgttgggcaagaatgggattgaggattatcttctctcagaaaggcattgtgaaggaattgagccagatctctctccctactgcaaaaccctattgtagtaaaaaagtcttctttactatcttaataaaacagatattgtgagattcaaaaaaaaaaaaaaaa

SEQ ID NO: 6 - protein, 335 aa, Homo sapiens
MAGSPTCLTLIYILWQLTGSAASGPVKELVGSVGGAVTFPLKSKVKQVDSIVWTFNTTPLVTIQPEGGTIIVTQNRNRERVDFPDGGYSLKLSKLKKNDSGIYYVGIYSSSLQQPSTQEYVLHVYEHLSKPKVTMGLQSNKNGTCVTNLTCCMEHGEEDVIYTWKALGQAANESHNGSILPISWRWGESDMTFICVARNPVSRNFSSPILARKLCEGAADDPDSSMVLLCLLLVPLLLSLFVLGLFLWFLKRERQEEYIEEKKRVDICRETPNICPHSGENTEYDTIPHTNRTILKEDPANTVYSTVEIPKKMENPHSLLTMPDTPRLFAYENVI

SEQ ID NO: 7 - DNA, 2111 bp, Homo sapiens
gggcagtcgtaggagactctgaaaaagcaaataaatcaatgttaaatcagaaatgtgaatgtagtaaggggctgaagagacaggggaagagaatacatgggaaaatattgaaaaggacagagtgatcaaaaagagcagggacatgggagcattgggcagcacactgggagccatttcactttatgctcttattgtatgattgagaaaaaaatgtccttagtggttaagtggcttttcaatgccacatcagacttgttccatagcagttgaattaggggaaggtgaataagttggaggttggtgacaaggagagaagctggaacagagaggagagtcagaaccagagggaaatgagagactgagtaggcatctcagggtttttgaaggagtggattttctttgttgcagtcaggggaggtttgtctgttggctgcagaaagaagtcagaatagagatatcgtggggtaggtttgtttggaacagaaatcaaagaccaatttttctgagagaaggaaataacatctgcaaatgatatgctgtttttgctacttccattgttagctgttctcccaggtgatggcaatgcagacgggctcaaggagcctctctccttccatgtcacctggatcgcatccttttacaaccattcctggaaacaaaatctggtctcaggttggctgagtgatttgcagactcatacctgggacagcaattccagcaccatcgttttcctgtgcccctggtccaggggaaacttcagcaatgaggagtggaaggaactggaaacattattccgtatacgcaccattcggtcatttgagggaattcgtagatacgcccatgaattgcagtttgaatatccttttgagatacaggtgacaggaggctgtgagctgcactctggaaaggtctcaggaagcttcttgcagttagcttatcaaggatcagactttgtgagcttccagaacaattcatggttgccatatccagtggctgggaatatggccaagcatttctgcaaagtgctcaatcagaatcagcatgaaaatgacataacacacaatcttctcagtgacacctgcccacgtttcatcttgggtcttcttgatgcaggaaaggcacatctccagcggcaagtgaagcccgaggcctggctgtcccatggccccagtcctggccctggccatctgcagcttgtgtgccatgtctcaggattctacccaaagcccgtgtgggtgatgtggatgcggggtgagcaggagcagcagggcactcagcgaggggacatcttgcccagtgctgatgggacatggtatctccgcgcaaccctggaggtggccgctggggaggcagctgacctgtcctgtcgggtgaagcacagcagtctagagggccaggacatcgtcctctactgggagcatcacagttccgtgggcttcatcatcttggcggtgatagtgcctttacttcttctgataggtcttgcgctttggttcaggaaacgctgtttctgttaagacacaccatgagcctcctcgtcacccttctccttttggggtgagagaccagcagcccaagggctccagacacacctgaacacatcgtgatgatgacgtcctctcaactctctttgtaaaaattttgttatttttgcttgtttctgattaatgattgtttgtcaatataagctcaatttaattttgcaggatttgttgttctgacctgggttctgggacttttaaattcaaattttatctccagatggaatggggtcctagcaacctccacatgttcaactattaatggatcatcaggcctgttttagatatcccttactccagagggccttccctgacttacaagtgggaagcagtctcttcctggtctgaactcccgccacattttagccgtactttgctaactgtgctcctcacttcctcttcttcattgcagttatttagatcccccctttccttctaatttttcagctccttcaatgcaaagtacatgtatttttaatatatgcatccctggtgaaggatcttgcctgcatgaaacatgttctcaataaaactctgtgttgaatttatgccaaaaaaaaaaaaaaaaaa

SEQ ID NO: 8 - protein, 327 aa, Homo sapiens
MLFLLLPLLAVLPGDGNADGLKEPLSFHVTWIASFYNHSWKQNLVSGWLSDLQTHTWDSNSSTIVFLCPWSRGNFSNEEWKELETLFRIRTIRSFEGIRRYAHELQFEYPFEIQVTGGCELHSGKVSGSFLQLAYQGSDFVSFQNNSWLPYPVAGNMAKHFCKVLNQNQHENDITHNLLSDTCPRFILGLLDAGKAHLQRQVKPEAWLSHGPSPGPGHLQLVCHVSGFYPKPVWVMWMRGEQEQQGTQRGDILPSADGTWYLRATLEVAAGEAADLSCRVKHSSLEGQDIVLYWEHHSSVGFIILAVIVPLLLLIGLALWFRKRCFC

SEQ ID NO: 9 - DNA, 1396 bp, Homo sapiens
ggcagttggaagagagaagaagtcactacagggtactgaggaaaagctttgctgaaattggagatcaaataccagctctgccagtaagaagttgcatctcccagtgaaatgctgctgctgccatttcaactgttagctgttctctttcctggtggtaacagtgaacatgccttccaggggccgacctcctttcatgttatccagacctcgtcctttaccaatagtacctgggcacaaactcaaggctcaggctggttggatgatttgcagattcatggctgggatagcgactcaggcactgccatattcctgaagccttggtctaaaggtaactttagtgataaggaggttgctgagttagaggagatattccgagtctacatctttggattcgctcgagaagtacaagactttgccggtgatttccagatgaaatacccctttgagatccagggcatagcaggctgtgagctacattctggaggtgccatagtaagcttcctgaggggagctctaggaggattggatttcctgagtgtcaagaatgcttcatgtgtgccttccccagaaggtggcagcagggcacagaaattctgtgcactaatcatacaatatcaaggtatcatggaaactgtgagaattctcctctatgaaacctgcccccgatatctcttgggcgtcctcaatgcaggaaaagcagatctgcaaagacaagtgaagcctgaggcctggctgtccagtggccccagtcctggacctggccgtctgcagcttgtgtgccatgtctcaggattctacccaaagcccgtgtgggtgatgtggatgcggggtgagcaggagcagcagggcactcagctaggggacatcctgcccaatgctaactggacatggtatctccgagcaaccctggatgtggcagatggggaggcggctggcctgtcctgtcgggtgaagcacagcagtttagagggccaggacatcatcctctactggagaaaccccacctccattggctcaattgttttggcaataatagtgccttccttgctccttttgctatgccttgcattatggtatatgaggcgccggtcatatcagaatatcccatgagccatcatcatgtctcctctcccattcgcaataagtaccaagaagcccaagatatcagcccaaaagtcaatcttatcatatttcaaatgattttcaaatttgatgaaatcagagttttcatgtattttaaaataaattattatttaaacatcagcaaaaaagtacttaaaactgtaaatttattatgagactgtactaacagtgtgattcaccctgattttacacacattaaaatgttagaaaaaatgtgtctcaaaataaatgaaatataatacatatgacttaa

SEQ ID NO: 10 - protein, 333 aa, Homo sapiens
MLLLPFQLLAVLFPGGNSEHAFQGPTSFHVIQTSSFTNSTWAQTQGSGWLDDLQIHGWDSDSGTAIFLKPWSKGNFSDKEVAELEEIFRVYIFGFAREVQDFAGDFQMKYPFEIQGIAGCELHSGGAIVSFLRGALGGLDFLSVKNASCVPSPEGGSRAQKFCALIIQYQGIMETVRILLYETCPRYLLGVLNAGKADLQRQVKPEAWLSSGPSPGPGRLQLVCHVSGFYPKPVWVMWMRGEQEQQGTQLGDILPNANWTWYLRATLDVADGEAAGLSCRVKHSSLEGQDIILYWRNPTSIGSIVLAIIVPSLLLLLCLALWYMRRRSYQNIP

SEQ ID NO: 11 - DNA, 6701 bp, Homo sapiens
aaagccctcagcctttgtgtccttctctgcgccggagtggctgcagctcacccctcagctccccttggggcccagctgggagccgagatagaagctcctgtcgccgctgggcttctcgcctcccgcagagggccacacagagaccgggatggccacctccatgggcctgctgctgctgctgctgctgctcctgacccagcccggggcggggacgggagctgacacggaggcggtggtctgcgtggggaccgcctgctacacggcccactcgggcaagctgagcgctgccgaggcccagaaccactgcaaccagaacgggggcaacctggccactgtgaagagcaaggaggaggcccagcacgtccagcgagtactggcccagctcctgaggcgggaggcagccctgacggcgaggatgagcaagttctggattgggctccagcgagagaagggcaagtgcctggaccctagtctgccgctgaagggcttcagctgggtgggcgggggggaggacacgccttactctaactggcacaaggagctccggaactcgtgcatctccaagcgctgtgtgtctctgctgctggacctgtcccagccgctccttcccagccgcctccccaagtggtctgagggcccctgtgggagcccaggctcccccggaagtaacattgagggcttcgtgtgcaagttcagcttcaaaggcatgtgccggcctctggccctggggggcccaggtcaggtgacctacaccacccccttccagaccaccagttcctccttggaggctgtgccctttgcctctgcggccaatgtagcctgtggggaaggtgacaaggacgagactcagagtcattatttcctgtgcaaggagaaggcccccgatgtgttcgactggggcagctcgggccccctctgtgtcagccccaagtatggctgcaacttcaacaatgggggctgccaccaggactgctttgaagggggggatggctccttcctctgcggctgccgaccaggattccggctgctggatgacctggtgacctgtgcctctcgaaacccttgcagctccagcccatgtcgtgggggggccacgtgcgtcctgggaccccatgggaaaaactacacgtgccgctgcccccaagggtaccagctggactcgagtcagctggactgtgtggacgtggatgaatgccaggactccccctgtgcccaggagtgtgtcaacacccctgggggcttccgctgcgaatgctgggttggctatgagccgggcggtcctggagagggggcctgtcaggatgtggatgagtgtgctctgggtcgctcgccttgcgcccagggctgcaccaacacagatggctcatttcactgctcctgtgaggagggctacgtcctggccggggaggacgggactcagtgccaggacgtggatgagtgtgtgggcccggggggccccctctgcgacagcttgtgcttcaacacacaagggtccttccactgtggctgcctgccaggctgggtgctggccccaaatggggtctcttgcaccatggggcctgtgtctctgggaccaccatctgggccccccgatgaggaggacaaaggagagaaagaagggagcaccgtgccccgtgctgcaacagccagtcccacaaggggccccgagggcacccccaaggctacacccaccacaagtagaccttcgctgtcatctgacgcccccatcacatctgccccactcaagatgctggcccccagtgggtccccaggcgtctggagggagcccagcatccatcacgccacagctgcctctggcccccaggagcctgcaggtggggactcctccgtggccacacaaaacaacgatggcactgacgggcaaaagctgcttttattctacatcctaggcaccgtggtggccatcctactcctgctggccctggctctggggctactggtctatcgcaagcggagagcgaagagggaggagaagaaggagaagaagccccagaatgcggcagacagttactcctgggttccagagcgagctgagagcagggccatggagaaccagtacagtccgacacctgggacagactgctgaaagtgaggtggccctagagacactagagtcaccagccaccatcctcagagctttgaactccccattccaaaggggcacccacatttttttgaaagactggactggaatcttagcaaacaattgtaagtctcctccttaaaggccccttggaacatgcaggtattttctacgggtgtttgatgttcctgaagtggaagctgtgtgttggcgtgccacggtggggatttcgtgactctataatgattgttactccccctcccttttcaaattccaatgtgaccaattccggatcagggtgtgaggaggccggggctaaggggctcccctgaatatcttctctgctcacttccaccatctaagaggaaaaggtgagttgctcatgctgattaggattgaaatgatttgtttctcttcctaggatgaaaactaaatcaattaattattcaattaggtaagaagatctggttttttggtcaaagggaacatgttcggactggaaacatttctttacatttgcattcctccatttcgccagcacaagtcttgctaaatgtgatactgttgacatcctccagaatggccagaagtgcaattaacctcttaggtggcaaggaggcaggaagtgcctctttagttcttacatttctaatagccttgggtttatttgcaaaggaagcttgaaaaatatgagaaaagttgcttgaagtgcattacaggtgtttgtgaagtcacataatctacggggctagggcgagagaggccagggatttgttcacagatacttgaattaattcatccaaatgtactgaggttaccacacacttgactacggatgtgatcaacactaacaaggaaacaaattcaaggacaacctgtctttgagccagggcaggcctcagacaccctgcctgtggccccgcctccacttcatcctgcccggaatgccagtgctccgagctcagacagaggaagccctgcagaaagttccatcaggctgtttcctaaaggatgtgtgaacgggagatgatgcactgtgttttgaaagttgtcattttaaagcattttagcacagttcatagtccacagttgatgcagcatcctgagattttaaatcctgaagtgtgggtggcgcacacaccaagtagggagctagtcaggcagtttgcttaaggaacttttgttctctgtctcttttccttaaaattgggggtaaggagggaaggaagagggaaagagatgactaactaaaatcatttttacagcaaaaactgctcaaagccatttaaattatatcctcattttaaaagttacatttgcaaatatttctccctatgataatgtagtcgatagtgtgcactctttctctctctctctctctctcacacacacacacacacacacacacacacacacagagacacggcaccattctgcctggggcactggaacacattcctgggggtcaccgatggtcagagtcactagaagttacctgagtatctctgggaggcctcatgtctcctgtgggctttttaccaccactgtgcaggagaacagacagaggaaatgtgtctccctccaaggccccaaagcctcagagaaagggtgtttctggttttgccttagcaatgcatcggtctctgaggtgacactctggagtggttgaagggccacaaggtgcagggttaatactcttgccagttttgaaatatagatgctatggttcagattgtttttaatagaaaactaaaggggcaggggaagtgaaaggaaagatggaggttttgtgcggctcgatggggcatttggaacttctttttaaagtcatctcatggtctccagttttcagttggaactctggtgtttaacacttaagggagacaaaggctgtgtccatttggcaaaacttccttggccacgagactctaggtgatgtgtgaagctgggcagtctgtggtgtggagagcagccatctgtctggccattcagaggattctaaagacatggctggatgcgctgctgaccaacatcagcacttaaataaatgcaaatgcaacatttctccctctgggccttgaaaatccttgcccttatcatttggggtgaaggagacatttctgtccttggcttcccacagccccaacgcagtctgtgtatgattcctgggatccaacgagccctcctattttcacagtgttctgattgctctcacagcccaggcccatcgtctgttctctgaatgcagccctgttctcaacaacagggaggtcatggaacccctctgtggaacccacaaggggagaaatgggtgataaagaatccagttcctcaaaaccttccctggcaggctgggtccctctcctgctgggtggtgctttctcttgcacaccactcccaccacggggggagagccagcaacccaaccagacagctcaggttgtgcatctgatggaaaccactgggctcaaacacgtgctttattctcctgtttatttttgctgttactttgaagcatggaaattcttgtttgggggatcttggggctacagtagtgggtaaacaaatgcccaccggccaagaggccattaacaaatcgtccttgtcctgaggggccccagcttgctcgggcgtggcacagtggggaatccaagggtcacagtatggggagaggtgcaccctgccacctgctaacttctcgctagacacagtgtttctgcccaggtgacctgttcagcagcagaacaagccagggccatggggacgggggaagttttcacttggagatggacaccaagacaatgaagatttgttgtccaaataggtcaataattctgggagactcttggaaaaaactgaatatattcaggaccaactctctccctcccctcatcccacatctcaaagcagacaatgtaaagagagaacatctcacacacccagctcgccatgcctactcattcctgaatttcaggtgccatcactgctctttctttcttctttgtcatttgagaaaggatgcaggaggacaattcccacagataatctgaggaatgcagaaaaaccagggcaggacagttatcgacaatgcattagaacttggtgagcatcctctgtagagggactccacccctgctcaacagcttggcttccaggcaagaccaaccacatctggtctctgccttcggtggcccacacacctaagcgtcatcgtcattgccatagcatcatgatgcaacacatctacgtgtagcactacgacgttatgtttgggtaatgtggggatgaactgcatgaggctctgattaaggatgtggggaagtgggctgcggtcactgtcggccttgcaaggccacctggaggcctgtctgttagccagtggtggaggagcaaggcttcaggaagggccagccacatgccatcttccctgcgatcaggcaaaaaagtggaattaaaaagtcaaacctttatatgcatgtgttatgtccattttgcaggatgaactgagtttaaaagaatttttttttctcttcaagttgctttgtcttttccatcctcatcacaagcccttgtttgagtgtcttatccctgagcaatctttcgatggatggagatgatcattaggtacttttgtttcaacctttattcctgtaaatatttctgtgaaaactaggagaacagagatgagatttgacaaaaaaaaattgaattaaaaataacacagtctttttaaaactaacataggaaagcctttcctattatttctcttcttagcttctccattgtctaaatcaggaaaacaggaaaacacagctttctagcagctgcaaaatggtttaatgccccctacatatttccatcaccttgaacaatagctttagcttgggaatctgagatatgatcccagaaaacatctgtctctacttcggctgcaaaacccatggtttaaatctatatggtttgtgcattttctcaactaaaaatagagatgataatccgaattctccatatattcactaatcaaagacactattttcatactagattcctgagacaaatactcactgaagggcttgtttaaaaataaattgtgttttggtctgttcttgtagataatgcccttctattttaggtagaagctctggaatccctttattgtgctgttgctcttatctgcaaggtggcaagcagttcttttcagcagattttgcccactattcctctgagctgaagttctttgcatagatttggcttaagcttgaattagatccctgcaaaggcttgctctgtgatgtcagatgtaattgtaaatgtcagtaatcacttcatgaatgctaaatgagaatgtaagtatttttaaatgtgtgtatttcaaatttgtttgactaattctggaattacaagatttctatgcaggatttaccttcatcctgtgcatgtttcccaaactgtgaggagggaaggctcagagatcgagcttctcctctgagttctaacaaaatggtgctttgagggtcagcctttaggaaggtgcagctttgttgtcctttgagctttctgttatgtgcctatcctaataaactcttaaacacattga

SEQ ID NO: 12 - protein, 652 aa, Homo sapiens
MATSMGLLLLLLLLLTQPGAGTGADTEAVVCVGTACYTAHSGKLSAAEAQNHCNQNGGNLATVKSKEEAQHVQRVLAQLLRREAALTARMSKFWIGLQREKGKCLDPSLPLKGFSWVGGGEDTPYSNWHKELRNSCISKRCVSLLLDLSQPLLPSRLPKWSEGPCGSPGSPGSNIEGFVCKFSFKGMCRPLALGGPGQVTYTTPFQTTSSSLEAVPFASAANVACGEGDKDETQSHYFLCKEKAPDVFDWGSSGPLCVSPKYGCNFNNGGCHQDCFEGGDGSFLCGCRPGFRLLDDLVTCASRNPCSSSPCRGGATCVLGPHGKNYTCRCPQGYQLDSSQLDCVDVDECQDSPCAQECVNTPGGFRCECWVGYEPGGPGEGACQDVDECALGRSPCAQGCTNTDGSFHCSCEEGYVLAGEDGTQCQDVDECVGPGGPLCDSLCFNTQGSFHCGCLPGWVLAPNGVSCTMGPVSLGPPSGPPDEEDKGEKEGSTVPRAATASPTRGPEGTPKATPTTSRPSLSSDAPITSAPLKMLAPSGSPGVWREPSIHHATAASGPQEPAGGDSSVATQNNDGTDGQKLLLFYILGTVVAILLLLALALGLLVYRKRRAKREEKKEKKPQNAADSYSWVPERAESRAMENQYSPTPGTDC

SEQ ID NO: 13 - DNA, 2664 bp, Homo sapiens
gctatctgaggaagttctggtagagagaagagctcaagagcatgggcagagtcagctcctgagtgggctgaacgctcccctcagctcctgcagtgctaattaagggagggagcagcggggagcttgcagtgaccaagagggtgttgaggctaggaggccacgataaacaggatacgataaaagtccttaaccaagacgcagatgggaagaagcgttagagcgagcagcactcacatctcaagaaccagcctttcaaacagtttccagagatggattatcctactttacttttggctcttcttcatgtatacagagctctatgtgaagaggtgctttggcatacatcagttccctttgccgagaacatgtctctagaatgtgtgtatccatcaatgggcatcttaacacaggtggagtggttcaagatcgggacccagcaggattccatagccattttcagccctactcatggcatggtcataaggaagccctatgctgagagggtttactttttgaattcaacgatggcttccaataacatgactcttttctttcggaatgcctctgaagatgatgttggctactattcctgctctctttacacttacccacagggaacttggcagaaggtgatacaggtggttcagtcagatagttttgaggcagctgtgccatcaaatagccacattgtttcggaacctggaaagaatgtcacactcacttgtcagcctcagatgacgtggcctgtgcaggcagtgaggtgggaaaagatccagccccgtcagatcgacctcttaacttactgcaacttggtccatggcagaaatttcacctccaagttcccaagacaaatagtgagcaactgcagccacggaaggtggagcgtcatcgtcatccccgatgtcacagtctcagactcggggctttaccgctgctacttgcaggccagcgcaggagaaaacgaaaccttcgtgatgagattgactgtagccgagggtaaaaccgataaccaatataccctctttgtggctggagggacagttttattgttgttgtttgttatctcaattaccaccatcattgtcattttccttaacagaaggagaaggagagagagaagagatctatttacagagtcctgggatacacagaaggcacccaataactatagaagtcccatctctaccagtcaacctaccaatcaatccatggatgatacaagagaggatatttatgtcaactatccaaccttctctcgcagaccaaagactagagtttaagcttattcttgacatgagtgcattagtaatgactcttatgtactcatgcatggatctttatgcaatttttttccactacccaaggtctaccttagatactagttgtctgaattgagttactttgataggaaaaatacttcattacctaaaatcatttttcatagaactgtttcagaaaacctgactctaactggtttatatacaaaagaaaacttactgtatcatataacagaatgatccaggggagattaagctttgggcaagggctatttaccagggcttaaatgttgtgtctagaattaagtatgggcataaactggcttctgaatccctttccagagtgttggatccatttccctggtcttggcctcactctcatgcaggctttcctcttgtgttggcaagatggctgccaactcttggcaattcatacatccttgtttctgtctggtagagagtttgcttctcaaatggagcaaacaaatttgattattttttcattgttaaataggcaacatgaccagaaaggatggaatggcttaagtaaactaagggttcacttctagagctgagaagcagggtcaaagcacaatactgggcaattcagagcatggttagaagaggaaaggggagtctcaaagctggagagtttaccaacaaatattgactgcagtgattaaccaagacatttttgttaactaaaaagtgaaatatgggatggattctagaaatggggtatctctgtccatacttctagaatccactctatcagcatagtccagaagaatacctggcagtagaagaaatgaatattcaagaggaagataaatgcgagagggcaatcctttactattctcatatttatttatctctcattctgtatagaattcttgccgccatcccaggtctagccttaggagcaaatgtagtagatagtcgaataataaataacttaatgttttggacatattttgtctacttttgagaattatttttaatatgtaaattctctcaaaagggtcaggcacctagttattattttttaatgattatgtgaaagttgaatataatataccactaaaagtgacagttgaaagtggtggcataggatggtagggtagaaatttgggagggaaaaaagaaattgggagggtacaggcaacaggagaaaggaatcaaaccacagaaaaatacaaagggaaacttctgcttcactattcagacaaagacagccctaatgacatcaccaacagtcaaagcaattagagaccatacctaatattgtttaaattctagatgtaggctaacaatgaaaagtatttgccaaactgaataaaactgtcatggttaccttgaaaaaaaaaaaaaaaaaaaaaaaaaaaaa

SEQ ID NO: 14 - protein, 336 aa, Homo sapiens
MDYPTLLLALLHVYRALCEEVLWHTSVPFAENMSLECVYPSMGILTQVEWFKIGTQQDSIAIFSPTHGMVIRKPYAERVYFLNSTMASNNMTLFFRNASEDDVGYYSCSLYTYPQGTWQKVIQVVQSDSFEAAVPSNSHIVSEPGKNVTLTCQPQMTWPVQAVRWEKIQPRQIDLLTYCNLVHGRNFTSKFPRQIVSNCSHGRWSVIVIPDVTVSDSGLYRCYLQASAGENETFVMRLTVAEGKTDNQYTLFVAGGTVLLLLFVISITTIIVIFLNRRRRRERRDLFTESWDTQKAPNNYRSPISTSQPTNQSMDDTREDIYVNYPTFSRRPKTRV

SEQ ID NO: 15 - DNA, 19 bp, Artificial
  primer
gtccacctcgacctgctct

SEQ ID NO: 16 - DNA, 19 bp, Artificial
  primer
cactggggcaggagaaact

SEQ ID NO: 17 - DNA, 19 bp, Artificial
  primer
tgggaaagcatcgctacac

SEQ ID NO: 18 - DNA, 20 bp, Artificial
  primer
gcactggagctggaaatagc

SEQ ID NO: 19 - DNA, 19 bp, Artificial
  primer
gggagaatccaagcaggac

SEQ ID NO: 20 - DNA, 20 bp, Artificial
  primer
ggaagctcctcgatctctga

SEQ ID NO: 21 - DNA, 20 bp, Artificial
  primer
tcgctgtttctacggtagcc

SEQ ID NO: 22 - DNA, 19 bp, Artificial
  primer
gggtgggtttgatgtaggc

SEQ ID NO: 23 - DNA, 21 bp, Artificial
  primer
cagccacaatcactcatcaga

SEQ ID NO: 24 - DNA, 19 bp, Artificial
  primer
agggtgggtttgctgtagg

SEQ ID NO: 25 - DNA, 18 bp, Artificial
  primer
ggagctcgtggtctcagg

SEQ ID NO: 26 - DNA, 21 bp, Artificial
  primer
cttggagtcggacttgttttg

SEQ ID NO: 27 - DNA, 19 bp, Artificial
  primer
gcaccatggtctcatccat

SEQ ID NO: 28 - DNA, 19 bp, Artificial
  primer
gtggctttggagaggttcc

SEQ ID NO: 29 - DNA, 18 bp, Artificial
  primer
ggagctcgtggtctcagg

SEQ ID NO: 30 - DNA, 20 bp, Artificial
  primer
gggtttgatgtaggctcctg

SEQ ID NO: 31 - DNA, 20 bp, Artificial
  primer
ggagataccgctgccactat

SEQ ID NO: 32 - DNA, 18 bp, Artificial
  primer
ggtgggtttgctgtaggc

SEQ ID NO: 33 - DNA, 23 bp, Artificial
  primer
ttcgaattcctcggtatatcttg

SEQ ID NO: 34 - DNA, 20 bp, Artificial
  primer
cacctccagttatgcgtctg

SEQ ID NO: 35 - DNA, 20 bp, Artificial
  primer
atgatgaagcctggaatgga

SEQ ID NO: 36 - DNA, 21 bp, Artificial
  primer
tcagagctttacggagctcat

SEQ ID NO: 37 - DNA, 20 bp, Artificial
  primer
gcctggaagagattcgtgac

SEQ ID NO: 38 - DNA, 22 bp, Artificial
  primer
cttcagagcttcgtagagagca

SEQ ID NO: 39 - DNA, 20 bp, Artificial
  primer
aagtgaggctggtgttggtt

SEQ ID NO: 40 - DNA, 21 bp, Artificial
  primer
cgtcttgtagctccacgtctt

SEQ ID NO: 41 - DNA, 19 bp, Artificial
  primer
agccacatcgctcagacac

SEQ ID NO: 42 - DNA, 19 bp, Artificial
  primer
gcccaatacgaccaaatcc

SEQ ID NO: 43 - DNA, 18 bp, Artificial
  primer
ccgcagcagaatggacag

SEQ ID NO: 44 - DNA, 18 bp, Artificial
  primer
gctcctggggtgtagatg

SEQ ID NO: 45 - DNA, 18 bp, Artificial
  primer
accgcagaaggtgcagac

SEQ ID NO: 46 - DNA, 21 bp, Artificial
  primer
ctggcttgatttcttcaggtg

SEQ ID NO: 47 - DNA, 18 bp, Artificial
  primer
ccgcagcagaatggacag

SEQ ID NO: 48 - DNA, 19 bp, Artificial
  primer
gcaggaggaacagaaagag

SEQ ID NO: 49 - DNA, 19 bp, Artificial
  primer
agccacatcgctcagacac

SEQ ID NO: 50 - DNA, 19 bp, Artificial
  primer
gcccaatacgaccaaatcc

(19) United States
(12) Patent Application Publication
(10) Pub. No.: US 2015/0057161 A1
(43) Pub. Date: Feb. 26, 2015

(54) HIGH-RESOLUTION TRANSCRIPTOME OF HUMAN MACROPHAGES

(71) Applicants: RHEINISCHE FRIEDRICH-WILHELMS-UNIVERSITÄT-BONN, Bonn (DE); BECTON DICKINSON AND COMPANY, Franklin Lakes, NJ (US)

(72) Inventors: Joachim Schultze, Konigswinter (DE); Michael Mallmann, Bonn (DE)

(73) Assignees: BECTON DICKINSON AND COMPANY, Franklin Lakes, NJ (US); RHEINISCHE FRIEDRICH-WILHELMS-UNIVERSITÄT-BONN, Bonn (DE)

(21) Appl. No.: 14/385,180

(22) PCT Filed: Mar. 13, 2013
(86) PCT No.: PCT/EP2013/055159

§ 371 Date: Sep. 15, 2014

(30) Foreign Application Priority Data
Mar. 14, 2012 (EP) 12159479.0

(51) Int. Cl. C12Q 1/68; C12N 5/0786

(52) U.S. Cl. CPC C12Q 1/6888; C12N 5/0645; C12Q 2600/158
USPC 506/2; 435/6.12; 435/6.11; 435/7.24; 435/372; 506/9; 506/13

(57) ABSTRACT

The invention is based on the finding of specific surface markers for M1-like (classically activated) and M2-like (alternatively activated) macrophages and provides for a method for the identification, characterization and isolation of M1-like and M2-like macrophages based on the abundance of said surface markers and for means for performing such method.

The invention is based on the finding of specific surface markers for M1-like and M2-like macrophages and provides for a method for the identification, characterization and isolation of M1-like (classically activated) and M2-like (alternatively activated) macrophages based on the abundance of said surface markers and for means for performing such method.

### BACKGROUND OF THE INVENTION

Macrophages represent resident phagocytic cells in the tissue and are involved in tissue homeostasis and induction of inflammatory reaction towards pathogens by use of their broad range of pattern-recognition receptors (Geissmann F. et al., Science 327(5966):656-661 (2010)). In context of the respective immune response, macrophages are polarized to specific functional properties, often referred to as M1-like and M2-like phenotype. Classically polarized M1-like macrophages can be induced by IFN-γ alone or together with LPS or TNF-α using M-CSF or GM-CSF (Martinez F. O. et al., The Journal of Immunology 177(10):7303-7311 (2006)). M1-like macrophages are effector cells of classical inflammatory immune responses exerting an IL-12^{high}, IL-23^{high} and IL-10^{low} phenotype with secretion of inflammatory cytokines IL-1β, IL-6 and TNF-α. They display a phenotype characterized by the expression of CD86, CD64, and CD16 (Biswas S. K., Mantovani A., Nat Immunol. 11(10):889-896 (2010); Mantovani A., Sica A., Curr Opin Immunol. 22(2):231-237 (2010)). In contrast, macrophages that are activated by other mechanisms than IFN-γ/LPS/TNF-α are grouped in the alternatively activated M2-like macrophage subset. Non-classically activated macrophages can be induced by cytokines including IL-4 and IL-13, but other stimuli have been described as well (Biswas S. K., Mantovani A., Nat Immunol. 11(10):889-896 (2010); Mantovani A., Sica A., Curr Opin Immunol. 22(2):231-237 (2010)). These cells share an IL-12^{low} and IL-23^{low} phenotype and express CD23. Over the last decade, phenotypic adaptations of macrophages to environmental stimuli have been linked to radical changes in transcriptional regulation mainly by applying microarray-based gene expression profiling (Martinez F. O. et al., The Journal of Immunology 177(10):7303-7311 (2006); Gustafsson C, Mjosberg J, Matussek A. et al., PLoS One. 3(4):e2078 (2008); Lehtonen A. et al., J Leukoc Biol. 82(3):710-720 (2007); Nau G. J. et al., Proc Natl Acad Sci USA 99(3):1503-1508 (2002)). In fact, a large amount of data covering transcriptional reprogramming of macrophages has been accumulated, albeit not always systematic (Martinez F. O. et al., The Journal of Immunology 177(10):7303-7311 (2006); Gustafsson C. et al., PLoS One. 3(4):e2078 (2008); Lehtonen A. et al., J Leukoc Biol. 82(3):710-720 (2007); Nau G. J. et al., Proc Natl Acad Sci USA 99(3):1503-1508 (2002); Heng T. S. et al., Nat Immunol. 9(10):1091-1094 (2008)). However, molecular mechanisms controlling transcriptional reprogramming in macrophages are far from understood and it has been suggested that integrative analyses of epigenomic and transcriptomic data will be required to better understand how macrophages integrate the information they receive from their respective microenvironment (Lawrence T., Natoli G., Nat Rev Immunol. 11(11):750-761 (2011)), enabling the identification of specific transcription factor combinations being responsible for cellular macrophage programs.

The introduction of RNA sequencing (RNA-seq) to interrogate whole transcriptomes has challenged previously established gene expression profiling studies (Ozsolak F., Milos P. M., Nature reviews Genetics 12(2):87-98 (2011); Wang Z, Gerstein M, Snyder M., Nature reviews Genetics 10(1):57-63 (2009); Marioni J. C. et al., Genome Res. 18(9):1509-1517 (2008)). Advantages assigned to RNA-seq over microarray analysis include increases in transcript quantity and quality, improved detection of alternative splicing events and gene fusion transcripts, and a larger dynamic range of detection (Ozsolak F., Milos P. M., Nature reviews Genetics 12(2):87-98 (2011); Wang Z. et al., Nature reviews Genetics 10(1):57-63 (2009); Marioni J. C. et al., Genome Res. 18(9):1509-1517 (2008)).

### SHORT DESCRIPTION OF THE INVENTION

To better understand polarization and integration of environmental signals by macrophages and to identify more specific markers for different functional states, high-resolution transcriptome data have been asked for (Murray P. J., Wynn T. A., Nat Rev Immunol. 11(11):723-737 (2011)). Using M1 and M2 polarization as models we applied RNA-seq and compared the information content with data derived by microarray analysis. We provide new insights into human macrophage biology and determine several new markers associated with classical and alternative macrophage polarization in humans.

The invention thus provides

(1) a method for identifying of, distinguishing between and isolating of M1-like (classically activated) and M2-like (alternatively activated) macrophages which comprises characterizing the macrophages based on the relative abundance of one or more of the specific M1-associated cell surface markers CD120b, TLR2 and SLAMF7, or of one or more of the specific M2-associated cell surface markers CD1a, CD1b, CD93 and CD226, respectively;
(2) a preferred embodiment of aspect (1) above, wherein the identifying of and distinguishing between the M1-like and M2-like macrophages is performed by an amplification or by a targeted resequencing of one or more of the specific M1-associated cell surface marker nucleic acids CD120b, TLR2 and SLAMF7 (SEQ ID NOs: 1, 3 and 5), or of one or more of the specific M2-associated cell surface marker nucleic acids CD1a, CD1b, CD93 and CD226 (SEQ ID NOs: 7, 9, 11 and 13), respectively, of the macrophage, notably by utilizing one or more primers derived from each of the marker genes, and a subsequent detection of the amplification/resequencing product;
(3) a preferred embodiment of aspect (1) above, wherein the identifying of and distinguishing between the M1-like and M2-like macrophages comprises hybridizing one or more probes selective for one of the specific M1-associated cell surface marker nucleic acids CD120b, TLR2 and SLAMF7 (SEQ ID NOs: 1, 3 and 5), or for one of the specific M2-associated cell surface marker nucleic acids CD1a, CD1b, CD93 and CD226 (SEQ ID NOs: 7, 9, 11 and 13), respectively, of the macrophage;
(4) a preferred embodiment of aspect (3) above, wherein the hybridization is performed on a hybridization array;
(5) a preferred embodiment of aspect (1) above, wherein the identifying of, distinguishing between and isolating of the M1-like and M2-like macrophages comprises contacting the macrophages with one or more binding molecules directed against the specific M1-associated cell surface marker protein CD120b, TLR2 and SLAMF7 (SEQ ID NOs: 2, 4 and 6), or with one or more binding molecules directed the specific M2-associated cell surface marker nucleic acids CD1a, CD1b, CD93 and CD226 (SEQ ID NOs: 8, 10, 12 and 14), respectively;
(6) a kit for performing the method according to (1) to (5) above, which comprises at least one reagent for identifying of, distinguishing between and isolating of M1-like macrophages and/or at least one reagent for identifying of, distinguishing between and isolating of M2-like macrophages, said reagents being selected from
(i) one or more primers derived from the marker genes as defined in (2) above,
(ii) one or more probes selective for the cell surface marker nucleic acids as defined in (3) above,
(iii) a hybridization array as defined in (4) above, or
(iv) one or more binding molecule as defined in (5) above;
(7) the use of antibodies selected from CD120b, TLR2 and SLAMF7 antibodies for identifying, characterizing and isolating M1-like macrophages;
(8) the use of antibodies selected from CD1a, CD1b, CD93 and CD226 antibodies for identifying, characterizing and isolating M2-like macrophages;
(9) a population of M1-like macrophages isolated by the method of (5) above; and
(10) a population of M2-like macrophages isolated by the method of (5) above.

### SHORT DESCRIPTION OF THE FIGURES

FIG. 1: Phenotypic characterization of human M1- and M2-like macrophages derived from CD14⁺ peripheral blood monocytes. Expression of typical macrophage lineage markers was determined by flow cytometry (left) of M1- and M2-like macrophages generated in the presence of GM-CSF (upper panel) or M-CSF (lower panel) with quantification shown in the graph at the right. Expression of (a) CD11b, (b) CD14, (c) CD68, (d) HLA-DR, (e) CD64, (f) CD86, and (g) CD23, respectively. *P<0.05 (Student's t-test). Numbers in plots indicate mean fluorescence intensity. Data are representative of nine independent experiments (a,b,d,e,f,g; mean and s.e.m.) or eight independent experiments (c; mean and s.e.m.), each with cells derived from a different donor.

FIG. 2: Microarray-based RNA fingerprinting of human M1- and M2-like macrophages. (a) Principle component analysis of human unpolarized (M0) and polarized (M1, M2) macrophages. (b) Unsupervised hierarchical clustering of human M0, M1-, and M2-like macrophages. (c) Visualization of known markers for human M1- and M2-like macrophages as a heatmap. Data were z-score normalized. (d) Left: network of genes highly expressed in M1-like macrophages (fold-change >2.0) in comparison to M0 macrophages identified by microarray analysis. Right: data for the comparison of M2-like versus M0 macrophages were loaded into the M1-network. (e) Right: network of genes highly expressed in M2-like macrophages (fold-change >1.65) in comparison to M0 macrophages identified by microarray analysis. Left: data for the comparison of M1-like versus M0 macrophages were loaded into the M2-network. All networks were generated using EGAN.

FIG. 3: Comparison of RNA-seq and microarray analysis. (a) Number of genes expressed in human M1- (left) and M2-like macrophages (right) as detected using RNA-Seq (black) and microarray analysis (white). (b) Correlation (Spearman) of mean expression values of M1- (left) and M2-like macrophages (right) using RNA-Seq and microarray analysis. (c-d) Comparison of differentially expressed genes detected using RNA-seq or microarray analysis (p<0.05). Differentially expressed genes as assessed by RNA-seq (black) or microarray analysis (white) were divided into groups by their relative expression in (c) M1 versus M2 or (d) M2 versus M1. (e) Gene expression in M1- versus M2-like macrophages as fold change versus fold change plot comparing microarray analysis with RNA-seq using all Refseq genes differentially expressed in RNA-seq. (f) Venn-diagram of differentially expressed genes between M1- and M2-like macrophages in RNA-seq (blue) and microarray analysis (red), (FC>2 p-value <0.05, diff>100 for microarray data). Fold-change-rank plots of genes detected as differentially expressed between M1- and M2-like macrophages (g) by microarray analysis (red) with overlay of values obtained by RNA-seq (blue) or (h) by RNA-seq (blue) with overlay of values obtained by microarray analysis (red). (i) Visualization of known markers for human M1- and M2-like macrophages from FIG. 2c as a heatmap using RNA-seq. Data were z-score normalized.

FIG. 4: Correlation of RNA-seq, microarray, qPCR, and flow cytometric analysis. (a-d) CD68, (e-h) CD64, and (i-l) CD23 expression in human M1- and M2-like macrophages. (a, e, i) Left, representative images of sequencing reads across the genomic loci of genes expressed in human macrophages. Pictures taken from the Integrative Genomics Viewer (IGV). The height of bars represents the relative accumulated number of 100-bp reads spanning a particular sequence. Gene maps (bottom portion of each panel, oriented 5′-3′ direction) are represented by thick (exons) and thin (introns) lines. Right, RPKM values by RNA-seq in M1- and M2-like macrophages. (b, f, j) Left, heatmaps presenting microarray results from M1- and M2-like macrophages from seven donors. Data were z-score normalized. Right, relative mRNA expression. (c, g, k) Relative mRNA expression by qPCR in M1- and M2-like macrophages. (d, h, l) Protein expression was determined by flow cytometry in human M1- and M2-like macrophages. Data are representative of three experiments (RNA-seq, mean and s.e.m.), seven experiments (microarray, mean and s.e.m.), at least seven experiments (qPCR; mean and s.e.m.), and nine experiments (flow cytometry), each with cells derived from a different donor. *P<0.05 (Student's t-test)

FIG. 5: Network analysis of RNA-seq data. (a) Network of genes highly expressed in M1-like macrophages (fold-change >4.0) identified by RNA-seq. (b) Data generated by microarray analysis were loaded into the M1-network established using RNA-seq. (c) Network of genes highly expressed in M2-like macrophages (fold-change >2.5) identified by RNA-seq. (d) Data generated by microarray analysis were loaded into the M2-network established using RNA-seq. All networks were generated using EGAN. (e) APOL1 and (f) LILRA1 expression in human M1- and M2-like macrophages. Left, representative images of sequencing reads across genes expressed in human macrophages as described in FIG. 4. Right, relative mRNA expression by qPCR in M1- and M2-like macrophages. Data are representative of three experiments (RNA-seq and qPCR; mean and s.e.m.) each with cells derived from a different donor. *P<0.05 (Student's t-test)

FIG. 6: Detection of alternative splicing in human macrophages. (a) Summarized expression of all PDLIM7 transcripts in human M1- and M2-like macrophages. Left, representative images of sequencing reads across genes expressed in human macrophages as described in FIG. 4. Right, RPKM values for PDLIM7 by RNA-seq in M1- and M2-like macrophages. (b) Expression of PDLIM7 as determined by microarray analysis using 3 different probes recognizing different parts of the PDLIM7 transcripts as depicted in (a). (c) Upper panel: representation of the 3 different mRNA transcripts from Refseq. Lower panel: abundance of the different transcripts as determined using Cuffdiff. (d) qPCR for the 3 different mRNA transcripts from Refseq in human M1- and M2-like macrophages. Data are representative of three experiments (RNA-seq), seven experiments (microarray analysis) or at least ten experiments (qPCR; mean and s.e.m.), each with cells derived from a different donor. *P<0.05 (Student's t-test)

FIG. 7: Identification of new macrophage polarization markers based on combined transcriptome analysis. Differentially expressed genes between M1- and M2-like macrophages of the human surfaceome were visualized as heatmaps for RNA-seq (left) and microarray analysis (right). Data were z-score normalized. (b-c) Expression of novel macrophage markers was determined by flow cytometry (left) of M1- and M2-like macrophages generated in the presence of GM-CSF with quantification shown in the graph at the right. Expression of (b) CD120b, TLR2, and SLAM7 as well as (c) CD1a, CD1b, CD93, and CD226. *P<0.05 (Student's t-test). Numbers in plots indicate mean fluorescence intensity. Data are representative of nine independent experiments (b,c; mean and s.e.m.) each with cells derived from a different donor.

FIG. 8: Phenotypic characterization of human M1-like macrophages derived from CD14⁺ peripheral blood monocytes. Expression of classical M1 markers after polarization of GM-CSF generated macrophages with IFN-γ, LPSu, TNF-α or IFN-γ and LPSu. Surface expression of lineage markers CD14 and CD11b as well as surface expression of the typical M1 markers CD86 and CD64 was assessed by flow cytometry.

FIG. 9: Comparison of RNA-seq and microarray analysis. Gene expression in M1- versus M2-like macrophages as fold change versus fold change plot comparing microarray analysis with RNA-seq using only Refseq genes differentially expressed in microarrays.

FIG. 10: Analysis of classical macrophage markers. (a) CD68, (b), CD64, and (c) CD23 expression in human M1- and M2-like macrophages. Representative images of sequencing reads across genes expressed in human macrophages for all three donors analyzed. Pictures taken from the Integrative Genomics Viewer (IGV). The height of bars represents the relative accumulated number of 100-bp reads spanning a particular sequence. Gene maps (bottom portion of each panel, oriented 5′-3′ direction) are represented by thick (exons) and thin (introns) lines.

FIG. 11: Detection of classical macrophage genes by RNA-seq. (a) IL-10 and (b) IL-18 expression in human M1- and M2-like macrophages. Left, expression as determined by microarray analysis using; middle, representative images of sequencing reads across genes expressed in human macrophages. Pictures taken from the Integrative Genomics Viewer (IGV). The height of bars represents the relative accumulated number of 100-bp reads spanning a particular sequence. Gene maps (bottom portion of each panel, oriented 5′-3′ direction) are represented by thick (exons) and thin (introns) lines. Right, relative mRNA expression by RNA-seq in M1- and M2-like macrophages. Data are representative of seven (microarrays, mean and s.d.) or three experiments (RNA-seq, mean and s.d.) each with cells derived from a different donor. *P<0.05 (Student's t-test), n.s.=not significant.

FIG. 12: Analysis of the apolipoprotein L family genes in M1- and M2-like macrophages. (a) APOL2, (b) APOL3, and (c) APOL6 expression in human M1- and M2-like macrophages. Left, relative expression as determined by RNA-seq; middle, representative images of sequencing reads across genes expressed in human macrophages. Pictures taken from the Integrative Genomics Viewer (IGV). The height of bars represents the relative accumulated number of 100-bp reads spanning a particular sequence. Gene maps (bottom portion of each panel, oriented 5′-3′ direction) are represented by thick (exons) and thin (introns) lines. Right, relative mRNA expression by qPCR in M1- and M2-like macrophages. Data are representative of three experiments (RNA-seq, mean and s.d. and qPCR, mean and s.e.m.) each with cells derived from a different donor. *P<0.05 (Student's t-test).

FIG. 13: Analysis of the leukocyte immunoglobulin-like receptor family genes in M1- and M2-like macrophages. (a) LILRA2, (b) LILRA3, (c) LILRA5, (d) LILRB1, and (c) LILRB3 expression in human M1- and M2-like macrophages. Left, relative expression as determined by RNA-seq; middle, representative images of sequencing reads across genes expressed in human macrophages. Pictures taken from the Integrative Genomics Viewer (IGV). The height of bars represents the relative accumulated number of 100-bp reads spanning a particular sequence. Gene maps (bottom portion of each panel, oriented 5′-3′ direction) are represented by thick (exons) and thin (introns) lines. Right, relative mRNA expression by qPCR in M1- and M2-like macrophages. Data are representative of three experiments (RNA-seq, mean and s.d. and qPCR, mean and s.e.m.) each with cells derived from a different donor. *P<0.05 (Student's t-test).

FIG. 14: Identification of new macrophage polarization markers based on combined transcriptome analysis. (a-b) Expression of novel M1- and M2-like macrophage markers on CD11b⁺CD14⁺ macrophages was determined by flow cytometry (left) of M1- and M2-like macrophages generated in the presence of M-CSF with quantification shown in the graph at the right. Expression of (a) CD120b, TLR2, and SLAM7 as well as (b) CD1a, CD1b, CD93, and CD226. *P<0.05 (Student's t-test). Numbers in plots indicate mean fluorescence intensity. Data are representative of nine independent experiments (b,c; mean and s.e.m.) each with cells derived from a different donor.

### DETAILED DESCRIPTION OF THE INVENTION

Macrophages are dynamic cells integrating signals from their microenvironment to develop specific functional responses. Microarray-based transcriptional profiling has established transcriptional reprogramming as an important mechanism for signal integration and cell function of macrophages yet current knowledge on transcriptional regulation is far from complete. RNA sequencing (RNA-seq) is ideally suited to fill this need but also to discover novel marker genes, an area of great need particularly in human macrophage biology. Applying RNA-seq, a high-resolution transcriptome profile of human macrophages under classical (M1-like) and alternative (M2-like) polarization conditions is provided and shows a dynamic range exceeding observations obtained by previous technologies, resulting in a more comprehensive understanding of the transcriptome of human macrophages. In addition, differential promoter usage, alternative transcription start sites, and different coding sequences for 57 gene loci in human macrophages were detected. Moreover, this approach led to the identification of novel M1-associated (CD120b, TLR2, SLAMF7) as well as M2-associated (CD1a, CD1b, CD93, CD226) cell surface markers.

Because of the enormous plasticity of human macrophages, the classification of polarization states on the basis of few cell surface markers will remain a substantial challenge (Murray P. J., Wynn T. A., Nat Rev Immunol. 11(11):723-737 (2011)). Here, we addressed how RNA-seq based high-resolution transcriptome data can be utilized to better understand the biology of macrophage polarization. We observed a significant increase in dynamic range in RNA-seq data resulting in a significantly higher number of genes determined to be significantly differentially expressed. This was true despite the fact that we used seven biological replicates for array analysis but only three samples for RNA-seq. A priori information based network analysis further supported that the increased information content of RNA-seq data uncovered novel aspects of macrophage biology, which was illustrated by the recognition of differential expression of numerous family members of two gene families, namely the apolipoprotein L family and leukocyte immunoglobulin-like receptors. APOLs constitute a new class of apolipoproteins expressed by macrophages as they serve as lytic factors against invading pathogens, e.g. African trypanosomes inducing programmed cell death as well as inhibiting intracellular infection by Leishmania (Pays E., Vanhollebeke B., Curr Opin Immunol. 21(5):493-498 (2009); Samanovic M. et al., PLoS Pathog. 5(1):e1000276 (2009)). LILRs have been associated with balancing the effects of Toll-like receptor signaling, suggesting an important role of LILRs both in the initiation but also cessation of inflammatory responses mediated by macrophages (Brown D. et al., Tissue Antigens. 64(3):215-225 (2004)). Another aspect enhancing our knowledge about the polarization biology of macrophages was the identification of several genes with differential usage of alternative promoters and transcription start sites as well as differential splicing variants between M1- and M2-like macrophages. As visualized for PDLIM7, an intracellular scaffold protein that contains a PDZ domain and three LIM domains linked to mitogenic signaling through actin cytoskeleton organization (Nakagawa N. et al., Biochemical and biophysical research communications 272(2):505-512 (2000)), regulating Tbx5 transcriptional activity (Camarata T. et al., Developmental biology 337(2):233-245 (2010)), and suppressing p53 activity (Jung C. R. et al., The Journal of clinical investigation 120(12):4493-4506 (2010)), RNA-seq revealed significant differences in splice variant usage for M1- and M2-like macrophages potentially linking p53 regulation with macrophage polarization (Matas D. et al., Cell death and differentiation 11(4):458-467 (2004)). Usage of splice variant-specific qPCR reactions supported these findings while this differential regulation was not revealed by microarray analysis. Altogether we detected differential promoter usage, transcription start site usage and splice variant usage in over 50 gene loci, a number that was surprisingly low taking into account that such mechanisms of transcriptional regulation have been suggested for the majority of gene loci in mammalian genomes (Kapranov P. et al., Nature reviews Genetics 8(6):413-423 (2007)).

While studies in other cell systems suggested that RNA-seq data will further improve cell characterization (Ozsolak F., Milos P. M., Nature reviews Genetics 12(2):87-98 (2011); Wang Z. et al., Nature reviews Genetics 10(1):57-63 (2009); Marioni J. C. et al., Genome Res. 18(9):1509-1517 (2008)), the direct assessment of the new technology in macrophage polarization was necessary to estimate its potential information gain. Both, increased dynamic range and the identification of transcripts that were missed by microarray analysis were major reasons for the discovery of novel genes associated with either M1- or M2-polarization. Nevertheless, despite a lower number of informative transcripts in the microarray data, 73% of the major M1-network was still revealed—at least when using transcripts defined to be enriched in M1-like macrophages. However, this rate dropped to only 54% in the M2-network and major hubs like MYC and TP53 where only revealed by RNA-seq data in M2-like macrophages. Overall these findings point towards an advantage of RNA-seq data, when the endpoint of the analysis is the identification of novel biological mechanisms.

An important aspect of genomic characterization is the identification of novel marker genes in macrophage polarization (Murray P. J., Wynn T. A., Nat Rev Immunol. 11(11):723-737 (2011)). When focusing on genes being part of the human surfaceome in most cases RNA-seq data revealed larger differences between M1-like and M2-like cells when compared to microarray data. Nevertheless, some genes only reached significant differential expression in the array data clearly pointing toward the necessity to include a large enough number of biological replicates also when applying RNA-seq. On the other hand, a subset of genes showed the well-known background noise effect in the microarray data resulting in non-significant differences between the two cell types. Irrespective of these different shortcomings of the two technologies, the overall differences between the two techniques in this defined gene space were less obvious suggesting that both technologies are well suited for cell surface marker identification. Taken together, we introduced several new marker genes for which we established FACS assays that can be used to distinguish between M1 and M2 polarization of macrophages and that can be combined with the analysis of common macrophage markers.

In the method of aspect (1) of the invention, the relative abundance of the M1-associated cell surface markers is higher in M1-like macrophages than in M2-like macrophages and the relative abundance of the M2-associated cell surface markers is higher in M2-like macrophages than in M1-like macrophages. It is preferred that the abundance of the cell surface marker in the respective M1-like or M2-like macrophage is at least 30%, more preferably at least 50% and most preferably at least 70% higher than in the other macrophage type.

The one or more primers employed in the amplification/resequencing employed in the method of aspect (2) of the invention are derived from the respective marker gene. Preferably said primers have at least 12, more preferably at least 15, most preferably at least 19 contiguous nucleotides of the respective marker nucleic acid sequence.

Similarly, the one or more probes employed in the method of aspect (3) of the invention are derived from the respective marker gene. Preferably said probes have a length that allows for a selective hybridization to the marker nucleic acid. The probe may also be labeled with a suitable marker molecule (e.g. with a fluorescence marker) to allow the detection of the resulting probe-surface marker nucleic acid complex.

Such probes may also be utilized in a hybridization array of aspect (4) of the invention.

The binding molecules utilized in aspect (5) of the invention include antibodies, preferably monoclonal antibodies. Moreover said binding molecules may be labeled with maker molecules, preferably fluorescence markers. Particular preferred binding molecules include the FITC-labeled CD1b, CD93, CD226 and anti-TLR2, PE-labeled CD120b and anti-SLAMF7, and PE-Cy5-labeled CD1a monoclonal antibodies. Further it is preferred that the method of aspect (5), notably if it is utilized to isolate the M1-like macrophages or M2-like macrophages, is performed on a FACS sorter.

The identification of novel marker genes distinguishing human M1-like and M2-like macrophages opens new avenues towards understanding the biology of differentially polarized macrophages. One of the M1-marker identified in this study, namely CD120b (TNFR2) has been linked to cell survival, activation and even proliferation in other cell types such as T cells (Faustman D., Davis M., Nat Rev Drug Discov. 9(6):482-493 (2010)). In contrast to TNFR1, TNFR2 preferentially leads to NFκB activation. Whether this is true in myeloid cells as well requires further investigation. However, earlier studies already suggested that production of TNF-α in macrophages might be interpreted as a phenotype-stabilizing feed-forward loop (Popov A. et al., The Journal of clinical investigation. 116(12):3160-3170 (2006)) and TNFR2 might actually play an important role in such a process.

SLAMF7 was originally identified as a NK cell-associated surface molecule (Boles K. S. et al., Immunol Rev. 181:234-249 (2001)). Subsequently, it was shown to be expressed on lymphocytes and monocytes (Murphy J. J. et al., Biochem J. 361(Pt 3):431-436 (2002)). More recently, a reduced expression on monocytes and NK cells with a simultaneous increase of SLAMF7 on B cells was observed in patients with lupus erythematosus (Kim J. R. et al., Clin Exp Immunol. 160(3):348-358 (2010)). The strongest link to SLAMF7 as an M1 marker gene comes from observations in intestine allograft rejection, demonstrating that tissue macrophages derived from patients rejecting the graft showed elevated levels of SLAMF7 (Ashokkumar C. et al., Am J. Pathol. 179(4):1929-1938 (2011)). It would be interesting to see if macrophages in other settings of transplant rejection are also enriched for this novel M1 marker gene. Considering the identification of single specific marker genes for macrophage polarization our findings clearly point to the necessity for multi-parameter analysis instead. This can be exemplified by the differential expression of CD1a and CD1b, two cell surface molecules that are mainly studied in context of antigen presentation by dendritic cells (Porcelli S. A., Modlin R. L., Annu Rev Immunol. 17:297-329 (1999)). Previous reports suggested upregulation of CD1 proteins on human monocytes by GM-CSF (Kasinrerk W. et al., J Immunol. 150(2):579-584 (1993)). However, we clearly present evidence that expression is induced in both M-CSF and GM-CSF driven macrophages and polarization towards M2-like macrophages is significantly increasing expression of CD1a and CD1b suggesting that they might be up-regulated on tissue macrophages in an M2-driving environment. This is similarly true for CD93, which was originally identified to be expressed on early hematopoietic stem cells and B cells (Greenlee-Wacker M. C. et al., Curr Drug Targets. (2011)). CD93 is involved in biological processes such as adhesion, migration, and phagocytosis (McGreal E. P. et al., J Immunol. 168(10):5222-5232 (2002); Nepomuceno R. R. et al., J Immunol. 162(6):3583-3589 (1999)). CD93 expressed on myeloid cells can be shed from the cell surface and the soluble form seems to be involved in differentiation of monocytes towards a macrophage phenotype (Jeon J. W. et al., J Immunol. 185(8):4921-4927 (2010)). Since soluble CD93 has been implicated in inflammatory responses, it will be important to further elucidate how polarization-induced differential expression of CD93 contributes to specific inflammatory responses. Another surprising finding is the differential expression of CD226 between human M1- and M2-like macrophages, a molecule initially shown to be involved in cytolytic function of T cells (Shibuya A. et al., Immunity. 4(6):573-581 (1996)). Subsequently, it could be shown that CD226 has additional functions including the regulation of monocyte migration through endothelial junctions (Reymond N. et al., J Exp Med. 199(10):1331-1341 (2004)). Similar to the other M2-associated markers, so far little is known about CD226 on polarized macrophages. Since CD226 expression levels on lymphocytes have been implicated in autoimmune diseases (Sinha S. et al., PLoS One. 6(7):e21868 (2011)) further research is necessary to understand its role in the myeloid compartment during such processes.

Overall, by using RNA-seq we introduce a high-resolution transcriptome analysis of human macrophages unraveling novel insights into macrophage polarization. While previously established transcriptome datasets addressing macrophage biology are still very suitable to assess important biological and medical questions, a deeper understanding of transcriptional regulation during macrophage polarization will require higher resolution that is provided by current and future RNA-seq technologies. Moreover, the novel cell surface markers will help to better understand macrophage programs and functions in human disease.

The Invention is further described in the following non-limiting Examples.

### EXAMPLES

### Materials and Methods

### Abbrevations

LPSu, ultrapure LPS; GEP, gene expression profiling; PCA, principle component analysis; RNA-seq, RNA sequencing technologies; MFI, mean fluorescence intensity; EGAN, exploratory gene association network; RPKM, Reads Per Kilobase of exon model per Million mapped reads; FC, fold change; TSS, transcription start sites; CDS, coding sequences.

Cell Isolation from Healthy Blood Donors:

Peripheral blood mononuclear cells (PBMC) were obtained by Pancoll (PAN-Biotech, Aidenbach, Germany) density centrifugation from buffy coats from healthy donors obtained following protocols accepted by the institutional review board at the University of Bonn (local ethics vote no. 045/09). Informed consent was provided for each specimen according to the Declaration of Helsinki. CD14⁺ monocytes were isolated from PBMC using CD14-specific MACS beads (Miltenyi Biotec) according to the manufacturers protocol (routinely >95% purity).

### Generation of Macrophages:

CD14⁺ monocytes were cultured in 6-well plates in RPMI1640 medium containing 10% FCS and differentiated into immature macrophages using GM-CSF (500 U/ml) or M-CSF (100 U/ml) for 3 days. Growth-factor containing medium was exchanged on day 3 and cells were polarized for 3 days with the following stimuli: IFN-γ (200 U/ml), TNF-α (800 U/ml), ultrapure LPS (LPSu, 10 μg/ml), IL-4 (1,000 U/ml), IL-13 (100 U/ml), or combinations thereof (all from Immunotools, Friesoythe, Germany).

### Monoclonal Antibodies and Flow Cytometry:

Cells were stained after FcR blockade incubating cells in PBS with 20% FCS for 10 minutes at 4° C. using the following monoclonal antibodies (all from Becton Dickinson (BD), BioLegend, or eBioscience): FITC-labeled CD1b, CD23, CD93, CD226, anti-HLA-DR, anti-TLR2; PE-labeled CD64, CD68, CD120b, anti-SLAMF7; PE-Cy5-labeled CD1a; PerCP-Cy5.5-labeled CD209; APC-labeled CD86; Pacific Blue-labeled CD11b; and APC-Cy7-labeled CD14 with matched isotype antibodies as controls. Intracellular staining of CD68 was performed using the BD Cytofix/Cytoperm kit (BD). Data were acquired on a LSR II (BD) and analyzed using FlowJo software (Tree Star).

### RNA Isolation:

5×10⁶-2×10⁷ macrophages were harvested, subsequently lysed in TRIZOL (Invitrogen) and total RNA was extracted according to the manufactures' protocol. The precipitated RNA was solved in RNAse free water. The quality of the RNA was assessed by measuring the ratio of absorbance at 260 nm and 280 nm using a Nanodrop 2000 Spectrometer (Thermo Scientific) as well as by visualization the integrity of the 28S and 18S band on an agarose gel.

### Quantitative PCR Conditions and Primer Sequences:

500 ng RNA was reverse transcribed using the Transcriptor First Strand cDNA Synthesis Kit (Roche Diagnostics). qPCR was performed using the LightCyclerTaqman master kit with GAPDH as reference on a LightCycler 480 II (Roche). qPCR primer sequences are summarized in Table 2.

Isoform specific PCR to identify alternative splicing events were performed using the Maxima SYBR Green/Fluorescein qPCR Master Mix (Fermentas). The relative enrichment of each isoform relative to GAPDH was calculated using the 2^{-ΔΔCT} method. qPCR primer sequences are listed in Table 3.

### Microarray-Based Transcriptional Profiling and Bioinformatic Analysis of Microarray Data:

Isolated RNA was further purified using the MinElute Reaction Cleanup Kit (Qiagen). Biotin labeled cRNA was generated using the TargetAmp Nano-g Biotin-aRNA Labeling Kit (Epicentre). Biotin labeled cRNA was hybridized to Human HT-12V3 Beadchips (Illumina) and scanned on an Illumina HiScanSQ system. Raw intensity data were exported with BeadStudio 3.1.1.0 (Illumina) and subsequently analysed using R (R Development Core Team. R: A Language and Environment for Statistical Computing (2011)). After quantile normalization non-informative genes (coefficient of variation <0.5) were excluded. From the resulting data sets we extracted a list of genes with a significant different expression in macrophage subtypes. Variable genes were plotted as heatmaps with hierarchical clustering using the correlation coefficient as a distance measure for the samples and the average of each cluster for cluster formation of the genes. Expression values are visualized with colors ranging from red (high expression) over white (intermediate expression) to blue (low expression). Principal component analysis (PCA) was performed using the “pcurve” package in R. Microarray data can be accessed under GSE35449.

### RNA-Seq and Data Analysis:

Sequencing and analysis were performed individually on M1-like and M2-like macrophages from 3 independent donors. Total RNA was converted into libraries of double stranded cDNA molecules as a template for high throughput sequencing using the Illumina CBot station and HiScanSQ following the manufacturer's recommendations using the Illumina TruSeq RNA Sample Preparation Kit. Shortly, mRNA was purified from 5-10 μg of total RNA using poly-T oligo-attached magnetic beads. Fragmentation was carried out using divalent cations under elevated temperature in Illumina proprietary fragmentation buffer. First strand cDNA was synthesized using random oligonucleotides and SuperScript II. Second strand cDNA synthesis was subsequently performed using DNA Polymerase I and RNase H. Remaining overhangs were converted into blunt ends via exonuclease/polymerase activities and enzymes were removed. After adenylation of 3′ ends of DNA fragments, Illumina PE adapter oligonucleotides were ligated to prepare for hybridization. In order to select cDNA fragments of preferentially 200 by in length the library fragments were separated on a 2% (w/v) agarose gel. The corresponding gel-fraction for each library was excised and purified using the QIAquick gel extraction kit (Qiagen). DNA fragments with ligated adapter molecules were selectively enriched using Illumina PCR primer PE1.0 and PE2.0 in a 15 cycle PCR reaction. Products were purified (QIAquick PCR purification kit) and quantified using the Agilent high sensitivity DNA assay on a Bioanalyzer 2100 system (Agilent). After cluster generation, 100 by paired-end reads were generated and analyzed using CASAVA 1.8. Alignment to the human reference genome hg19 from UCSC was performed stepwise. First, all reads passing the chastity filter were aligned to the reference genome. Next, reads were aligned to the RNA reference transcriptome. Based on these alignments the numbers of reads aligning to intragenic regions, or intergenic regions, respectively, were calculated. In addition the numbers of reads mapping to exonic and intronic regions as well as to splice sites were calculated based on the UCSC annotation file. Reads per kilobase of exon model per million mapped reads (RPKM) values for Refseq genes were established using CASAVA 1.8. In order to identify reads spanning altered splicing events or gene fusion breakpoints we also analyzed reads using TopHat and Bowtie. Results were further processed using Cufflinks and Cuffdiff (Trapnell C. et al., Nature biotechnology 28(5):511-515 (2010)).

### A Priori Information-Based Network Analysis Using EGAN Software:

To visualize connectivity between genes in high-throughput datasets contextual network graphs were generated based on a priori knowledge stored in literature, pathway, interaction, or annotation term databases by EGAN (exploratory gene association network) Paquette J., Tokuyasu T., Bioinformatics 26(2):285-286 (2010). To visualize the transcriptional regulation of genes enriched in M1 respectively M2, array data were used and fold change differences calculated using unpolarized macrophages as comparison. Genes with a FC>2 for M1 and FC>1.65 for M2 were visualized; represented is the major network. Using the network topology established for M1-like macrophages the expression values for M2-like macrophages were plotted and vice versa. For comparison of network components and density between RNA-seq and array data, the network was first visualized for the RNA-seq data (FC>4 for M1 and FC>2.5 for M2). Keeping the network topology, genes were marked according to their fold change when visualizing the array-based network. Graphs for genes enriched in M1 respectively in M2 were generated independently.

### Statistical Analysis:

Student's t-tests were performed with SPSS 19.0 software.

### Example 1

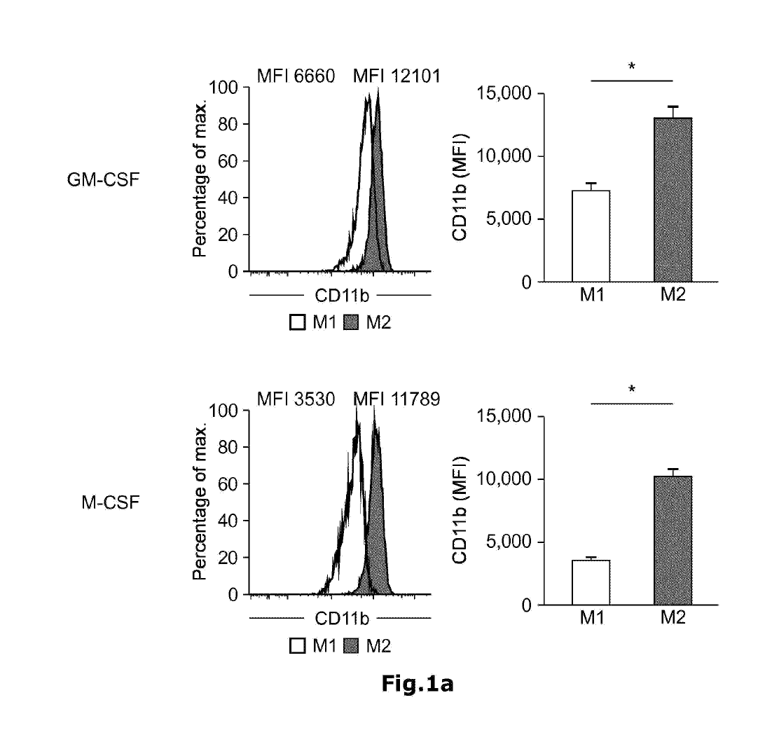

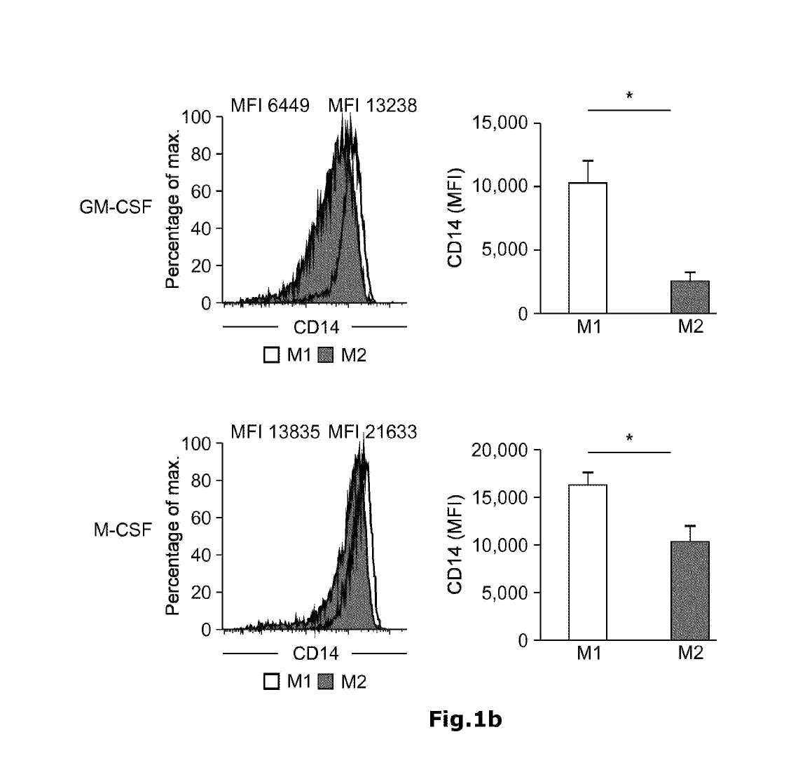

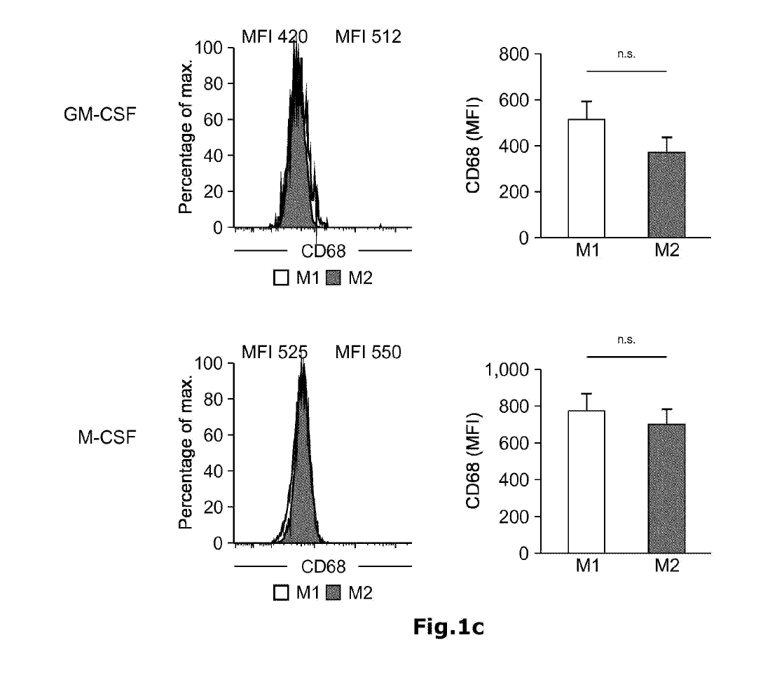

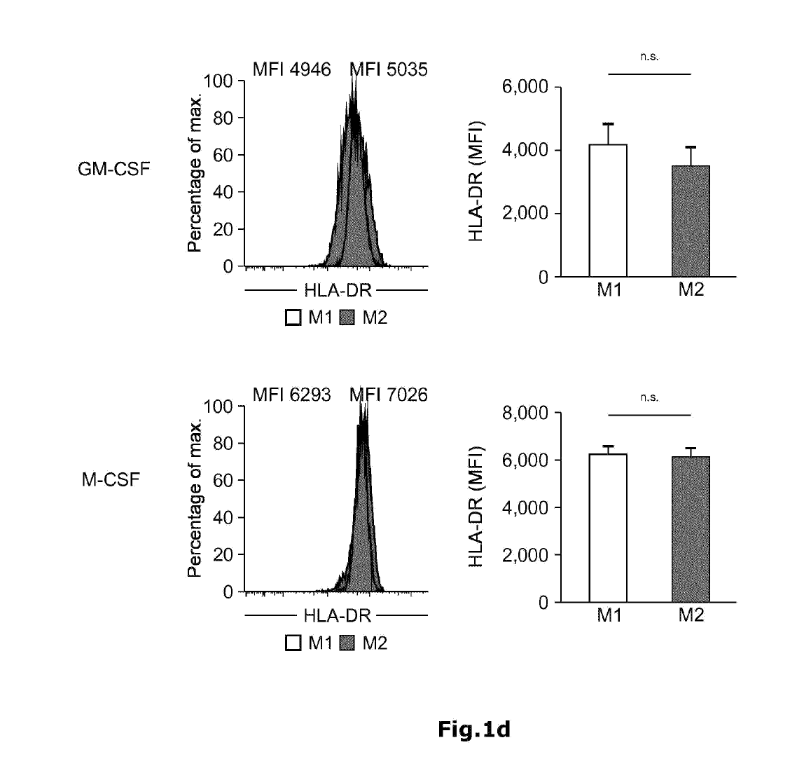

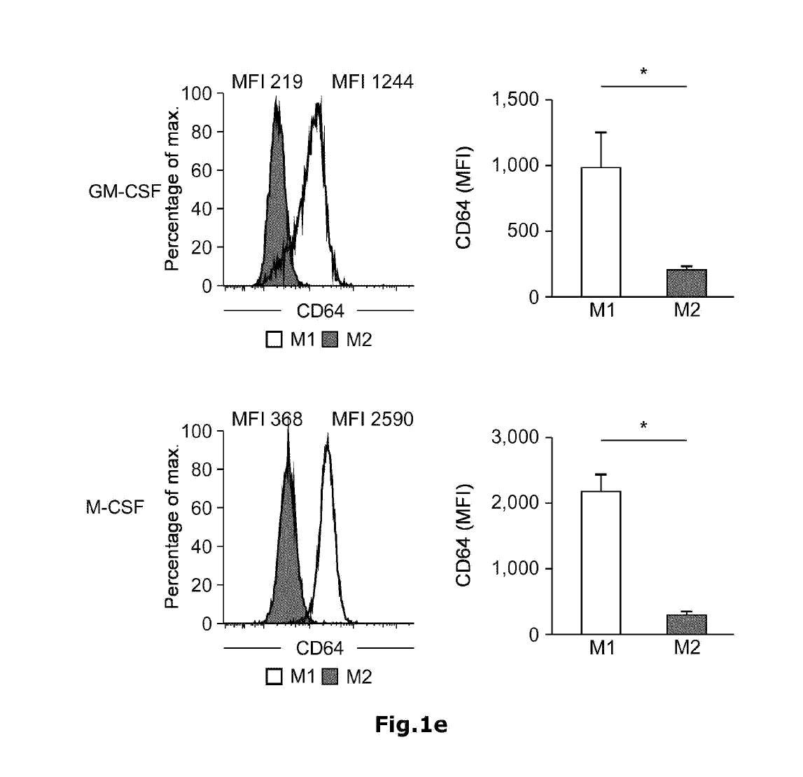

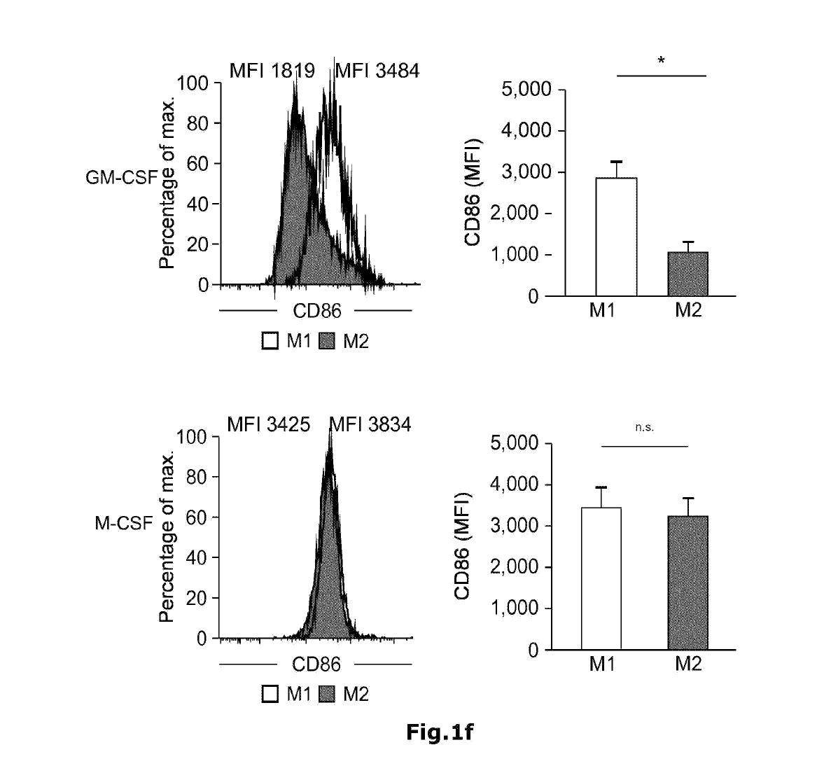

Generation of human M1- and M2-like macrophages as a model system. To establish a high-resolution transcriptome of human macrophages as a result of specific polarization signals, we used classical (M1-like) and alternative (M2-like) polarization of human macrophages as a model system. Since both M-CSF and GM-CSF have been described to differentiate macrophages from blood-derived CD14⁺ monocytes, we first compared the two different stimuli in respect to macrophage polarization and used expression of well-known macrophage markers as the initial readout (Martinez F. O. et al., The Journal of Immunology 177(10):7303-7311 (2006); Hamilton J. A., Nat Rev Immunol. 8(7):533-544 (2008)). For classical polarization we primarily used IFN-γ as the model stimulus and IL-4 for alternative polarization. When assessing the macrophage surface marker CD11b, the total percentage of CD11b⁺ cells under M1 and M2 polarization conditions was similar while the MFI was slightly higher in M2-like macrophages independent of the usage of GM-CSF or M-CSF (FIG. 1a). Further, we observed high expression of CD14 on all cells under M1 and M2 polarizing conditions irrespective of GM-CSF or M-CSF differentiation with a higher CD14 expression in M1-like macrophages (FIG. 1b). For both classical macrophage markers CD68 and MHC class II molecules (FIG. 1c and 1d) we observed no differences in all four conditions tested. Of note, when the IL-4 signal was provided to monocytes from the beginning of the differentiation period, immature dendritic cells were generated with a typical loss of macrophage markers such as CD14 or CD68 (data not shown).

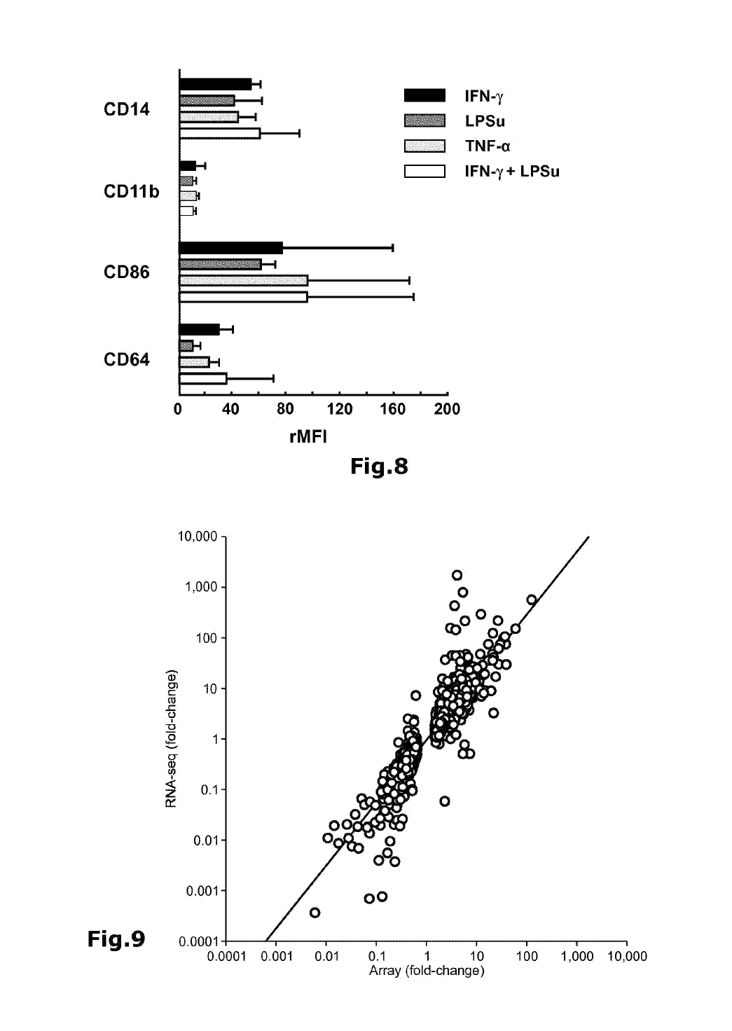

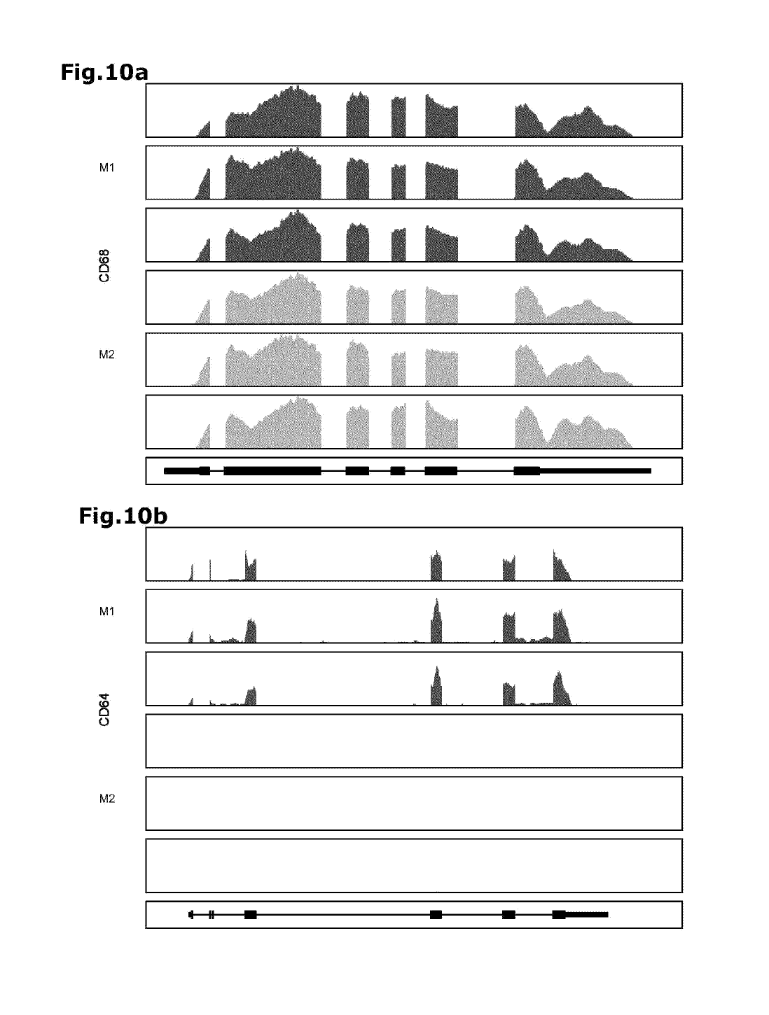

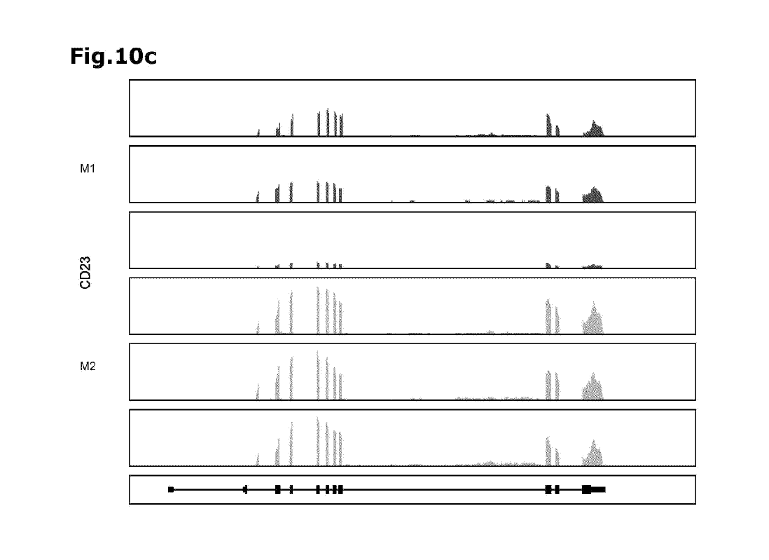

When assessing markers previously associated with M1 or M2 polarization (Mantovani A. et al., Trends Immunol. 23(11):549-555 (2002)), a selective induction of the M1 marker CD64 in M1-like macrophages was observed in cultures differentiated with both GM-CSF and M-CSF (FIG. 1e) while CD86 only showed an M1-specific expression in GM-CSF differentiated cells (FIG. 1f). Assessment of these markers following other M1-associated polarization signals, e.g. LPS, TNF-α or combinations thereof resulted in comparable results (FIG. 8). Inversely, strong induction of the M2-marker CD23 was observed in IL-4 polarized macrophages with significantly higher induction in GM-CSF polarized M2-like macrophages (FIG. 1g). For further experiments we therefore differentiated monocytes with GM-CSF for 3 days prior to polarization with either IFN-γ or IL-4 as the model signals.

### Example 2

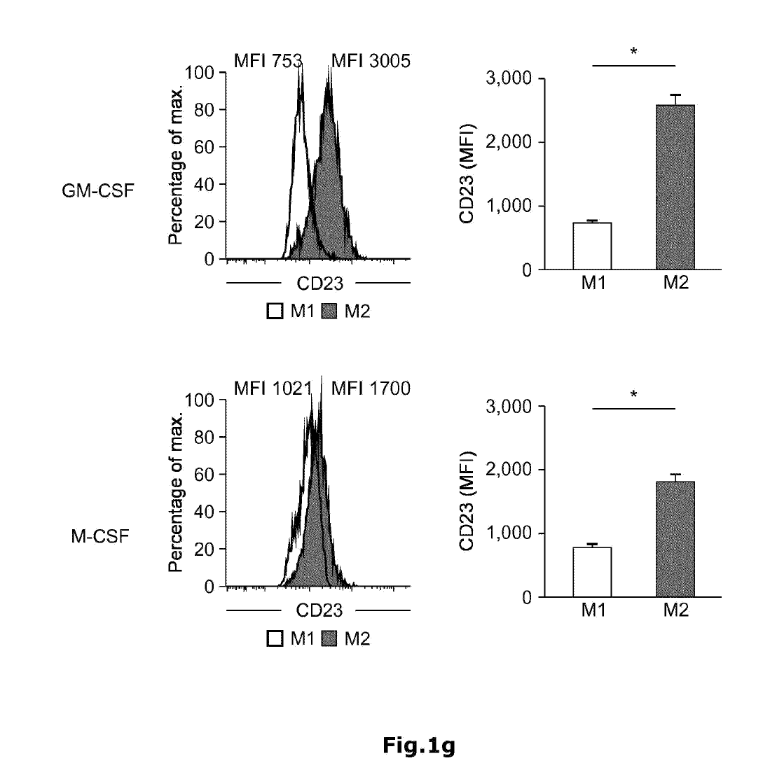

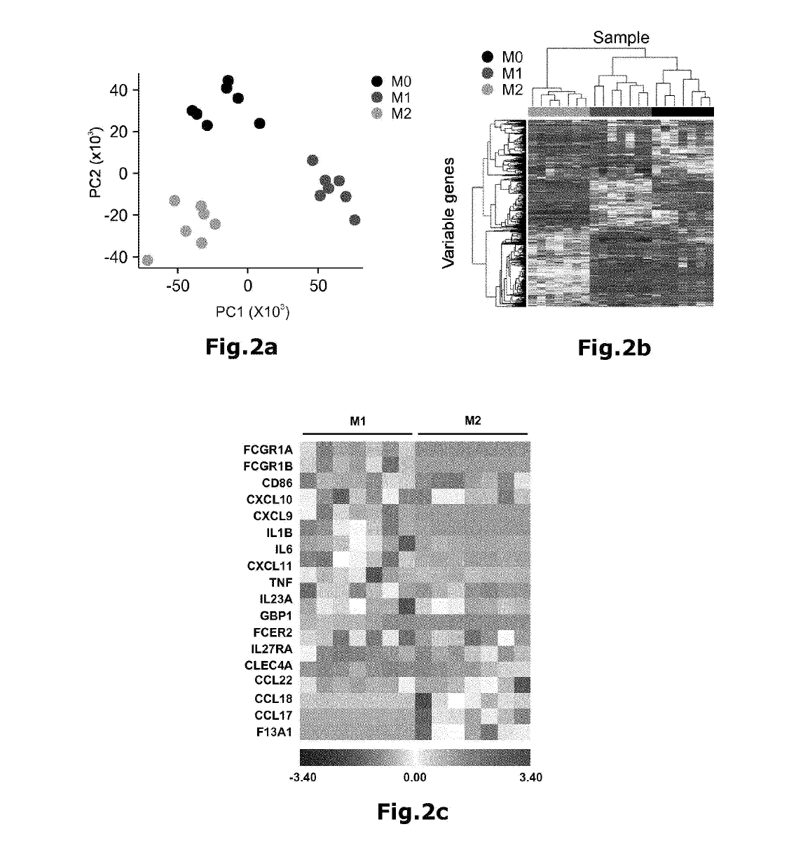

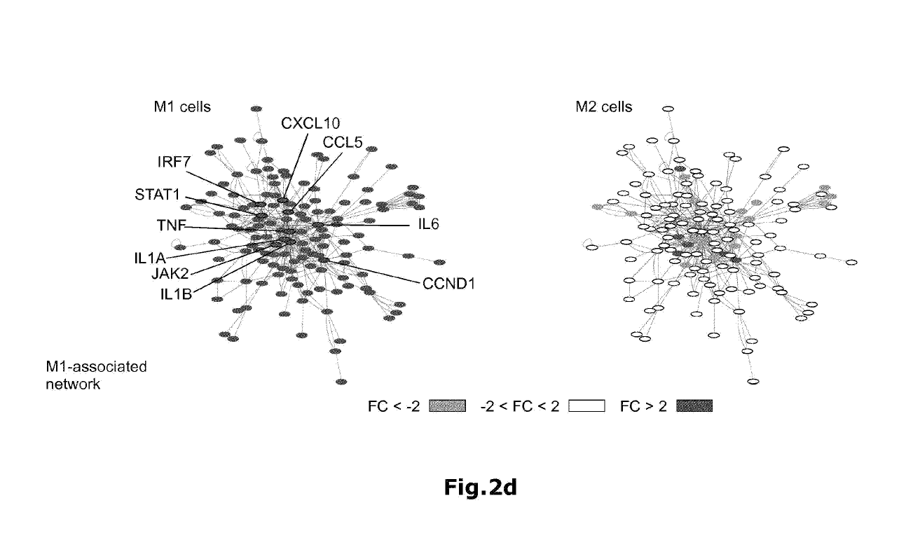

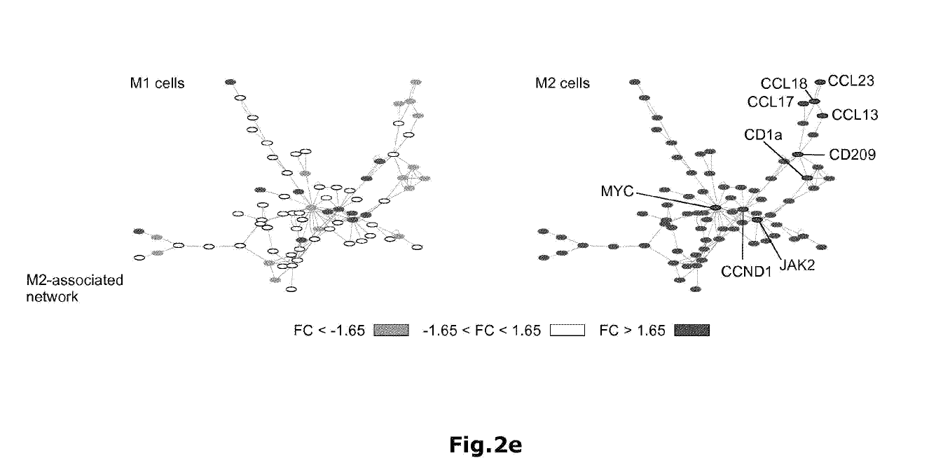

Characterization of M1- and M2-like macrophages by microarray-based gene expression profiling. Most recently it has been suggested that assessment of macrophage polarization in humans cannot solely rely on few cell surface markers but should be accommodated by gene expression profiling (Murray P. J., Wynn T. A., Nature reviews Immunology 11(11):723-737 (2011)). Using one of the most recent array generations, gene expression profiling was performed on unpolarized and polarized macrophages derived from seven healthy donors. To determine sample relationships, PCA (FIG. 2a) and hierarchical clustering (FIG. 2b) based on variable genes were performed and showed segregation of the samples by polarization state. Comparing our data with publically available datasets from M1- and M2-like macrophages generated with earlier array versions we observed concordant gene expression patterns (data not shown) (Martinez F. O. et al., The Journal of Immunology 177(10):7303-7311 (2006)). Heatmap visualization of known M1- and M2-like macrophage markers (FIG. 2c) further demonstrated that the genes encoding for the surface molecules FCGR1A and FCGR1B (both representing CD64) and CD86, the cytokine/chemokine genes CXCL10, CXCL9, IL-1B, IL-6, CXCL11, TNF, IL-23A, and the genes encoding for the intracellular protein GBP1 were increased in M1-like macrophages, results similar to what has been previously reported for M1 polarization (Martinez F. O. et al., The Journal of Immunology 177(10):7303-7311 (2006); Mantovani A. et al., Immunity 23(4):344-346 (2005); Mosser D. M., Edwards J. P., Nat Rev Immunol. 8(12):958-969 (2008)). Inversely, M2-associated genes encoding for the surface molecules FCER2 (CD23), IL27RA, and CLEC4A, the chemokine genes CCL22, CCL18, and CCL17, and the intracellular protein F13A1 were increased in the M2-like macrophages (Mantovani A. et al., Immunity 23(4):344-346 (2005); Mosser D. M., Edwards J. P., Nat Rev Immunol. 8(12):958-969 (2008); Wirnsberger G. et al., Eur J Immunol. 36(7):1882-1891 (2006)).

To further illustrate differential macrophage polarization, we generated a priori knowledge based M1-associated (FIG. 2d) and M2-associated (FIG. 2e) networks applying EGAN (Paquette J., Tokuyasu T., Bioinformatics 26(2):285-286 (2010)) using genes significantly upregulated in M1- (FC>2) respectively M2-polarized cells (FC>1.65). When applying expression values from the comparison of M2-like with M0 macrophages on the M1-associated network, the vast majority of genes showed either no change or even a reduction in expression, likely to represent an active repression of M1-associated genes in M2-like macrophages (FIG. 2d). Only few genes showed a simultaneous increase in expression, and these genes represented common cell cycle associated genes. Similarly, members of the M2-associated network were mostly not changed or even reduced in M1-like macrophages (FIG. 2e).

### Example 3

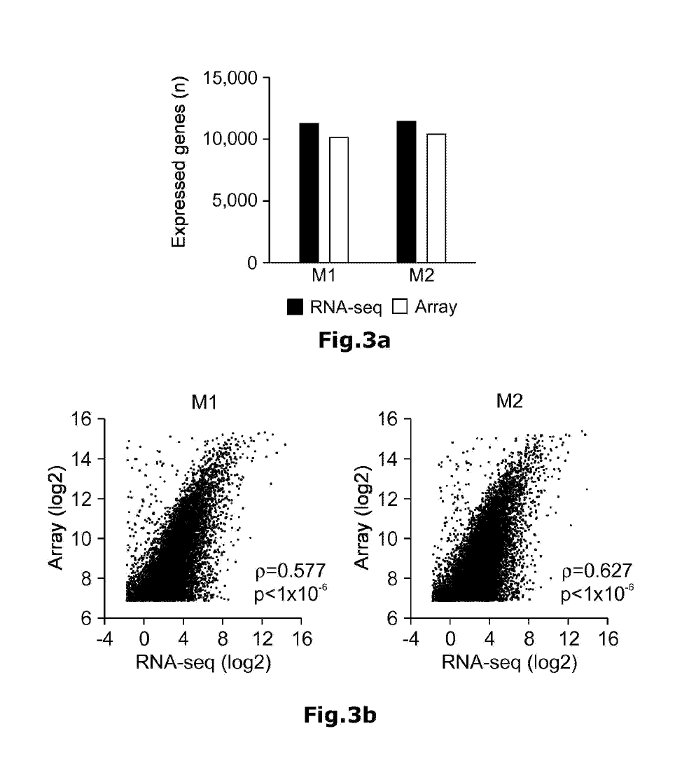

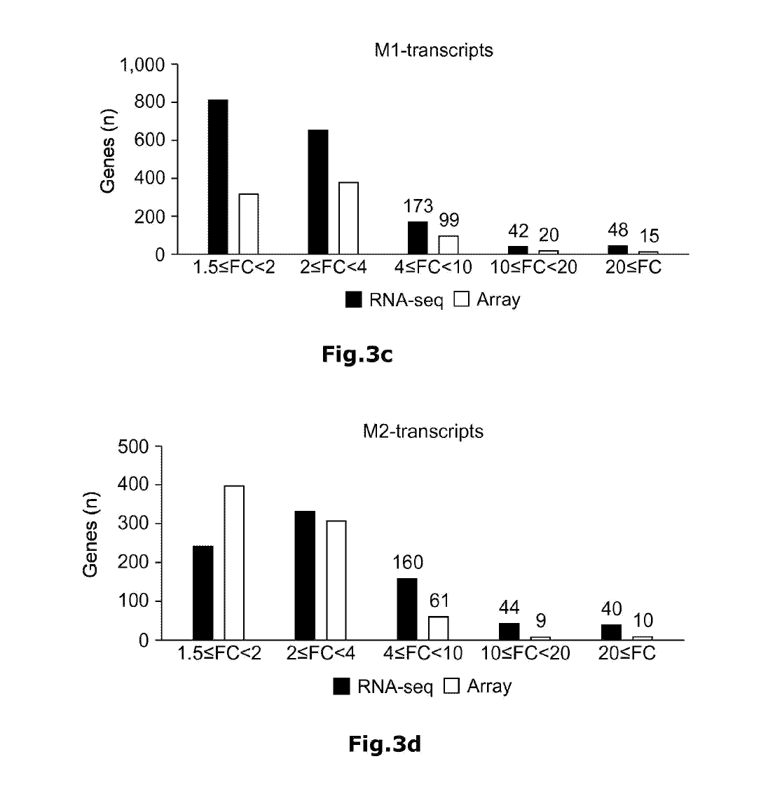

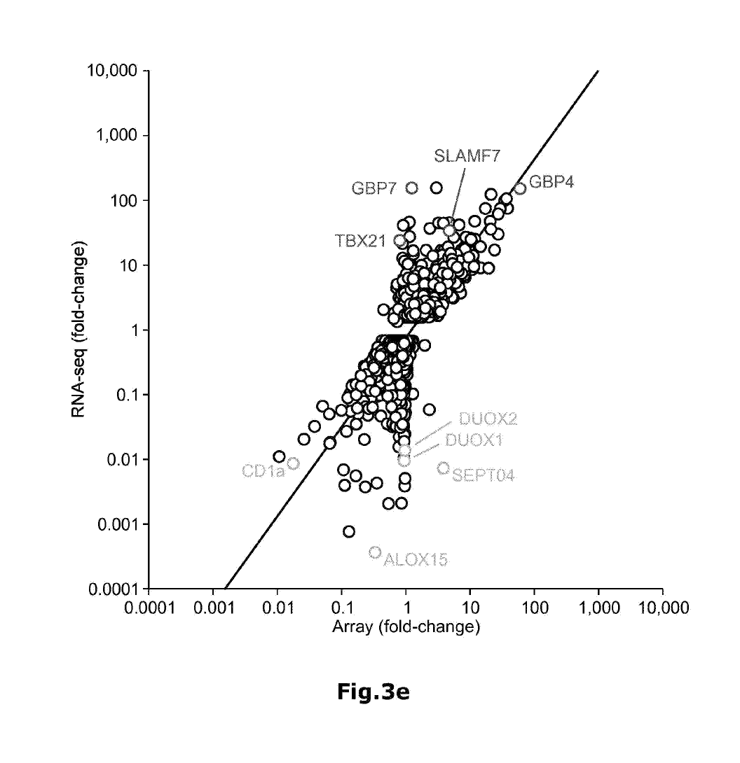

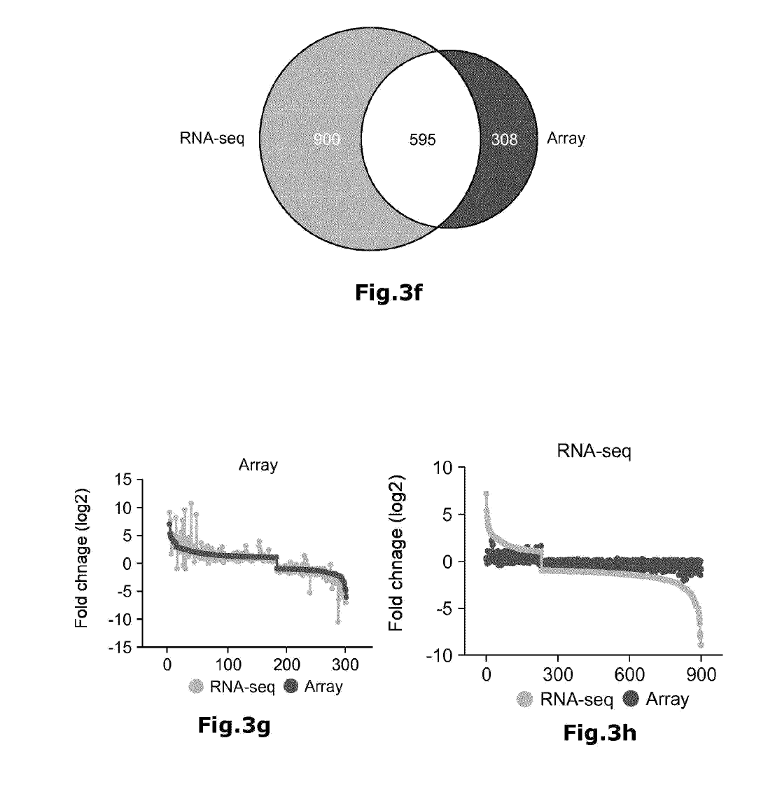

Increase in overall transcriptome information by RNA-seq. Gene expression profiling using microarrays has recently been suggested to be inferior to newer sequencing based technologies in providing genome-wide transcriptome information (Wang Z. et al., Nature reviews Genetics 10(1):57-63 (2009)). To address the potential information increase for macrophages, RNA-seq was performed on in vitro generated M1- and M2-like macrophages. After quality filtering, we obtained 15.0±2.8 million and 20.4±9.2 million reads for the M1- and M2-like macrophage cDNA libraries (Table 1). Consistent with RNA-seq data obtained from other eukaryotic cells (Ramskold D. et al., PLoS computational biology 5(12):e1000598 (2009)) the majority of sequencing reads for M1- and M2-like macrophages mapped to annotated exons (Refseq transcripts). The remaining reads mapped to exon-intron boundaries, introns, or other uncharacterized intergenic regions (Table 1). RPKMs are measures of individual transcript abundance in RNA-seq datasets and have been shown to be highly accurate across multiple cell types (Mortazavi A. et al., Nature methods 5(7):621-628 (2008)). We used CASAVA to assign RPKMs. To compare RNA-seq and microarray data we cross-annotated RNA-seq and microarray data using HGNC symbols. In human M1- and M2-like macrophages, 11317 and 11466 Refseq genes were expressed applying a previously defined optimal threshold (0.3 RPKM) for gene expression (FIG. 3a) (Ramskold D. et al., PLoS computational biology 5(12):e1000598 (2009)). The present call rate for Refseq genes for M1-(n=10155) and M2-like macrophages (n=10418) was only slightly lower when using microarrays (FIG. 3a). Furthermore, when assessing the levels of mRNA expression on a global scale a high correlation between RNA-seq and microarray data—similar to other cells (Mortazavi A. et al., Nature methods. 5(7):621-628 (2008))—was observed for M1- and M2-like macrophages (FIG. 3b).

### Example 4

RNA-seq reveals differential expression at significantly higher resolution. RNA-seq showed a significantly increased dynamic range over background mainly due to significantly lower background levels. This suggested that the assessment of differential expression using RNA-seq might lead to an improved resolution in comparison to array-based data. Applying standard filter criteria (FC 1.5, p<0.05, RPKM 0.3) revealed a total of 1736 genes elevated in M1- versus M2-like macrophages by RNA-seq, while 834 genes were recognized by array analysis (FIG. 3c). Similarly, 822 genes were identified as being elevated in M2- versus M1-like macrophages by RNA-seq, while 786 genes were detected by array analysis (FIG. 3d). More importantly, when categorizing differentially expressed genes according to their level of differential expression, RNA-seq data clearly revealed up to 4-fold more genes with FC>4 for M1- and M2-associated genes (FIGS. 3c and d), which was similarly true for M1-associated genes at lower levels (1.5<FC<4). To reveal potential reasons for this difference on the single-gene level we utilized FC-FC plotting, correlating RNA-seq and array-based data for individual genes (FIG. 3e). The majority of genes showed similar behavior in both RNA-seq and microarray experiments, albeit the relative differences were higher in RNA-seq data (FIG. 3e). Altogether, we observed a considerable increase in the dynamic range of fold-differences in RNA-seq data with differences spanning six orders of magnitude in contrast to only four orders of magnitude in the microarray data (FIGS. 3e and 9). In addition, there was a significant number of genes showing differential expression in RNA-seq data (e.g. DUOX1, DUOX2, TBX21, GBP7) but not in the array data suggesting that the array data are not informative for this class of genes. As anticipated, when using Venn diagrams with a defined cutoff (−2≧FC≧2, p<0.05, RPKM≧0.3) for data presentation (FIG. 3f), both RNA-seq and array data revealed 595 genes to be differentially expressed, but RNA-seq revealed 900 additional genes. Surprisingly, 308 genes were classified as being differentially expressed by array analysis alone (FIG. 3f). When further assessing these genes, it became apparent that these genes show a similar trend in the RNA-seq data but these differences did not yet reach statistical significance (FIG. 3g). In contrast, genes only identified by RNA-seq, clearly showed no differences when assessed by array analysis (FIG. 3h). Visualization of typical marker genes as depicted for array data in FIG. 2c demonstrated a comparable differentiation of M1- and M2-like macrophages when assessed by RNA-seq (FIG. 3i).

### Example 5

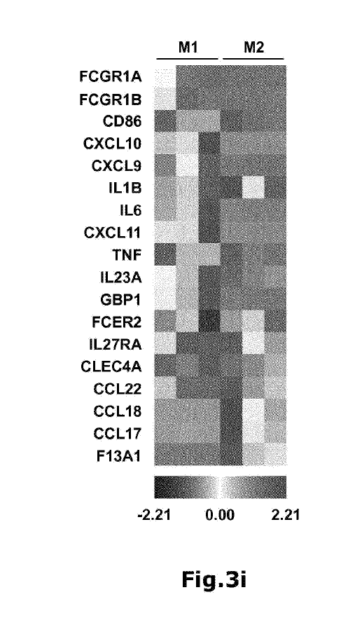

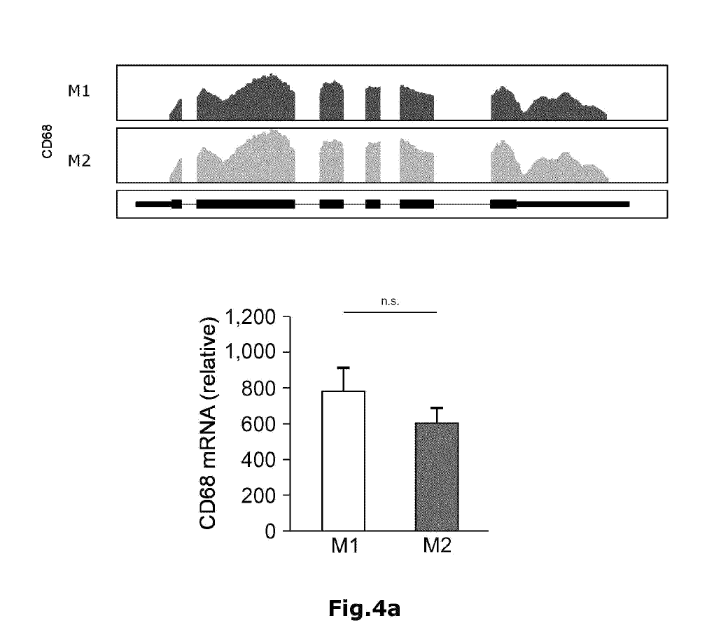

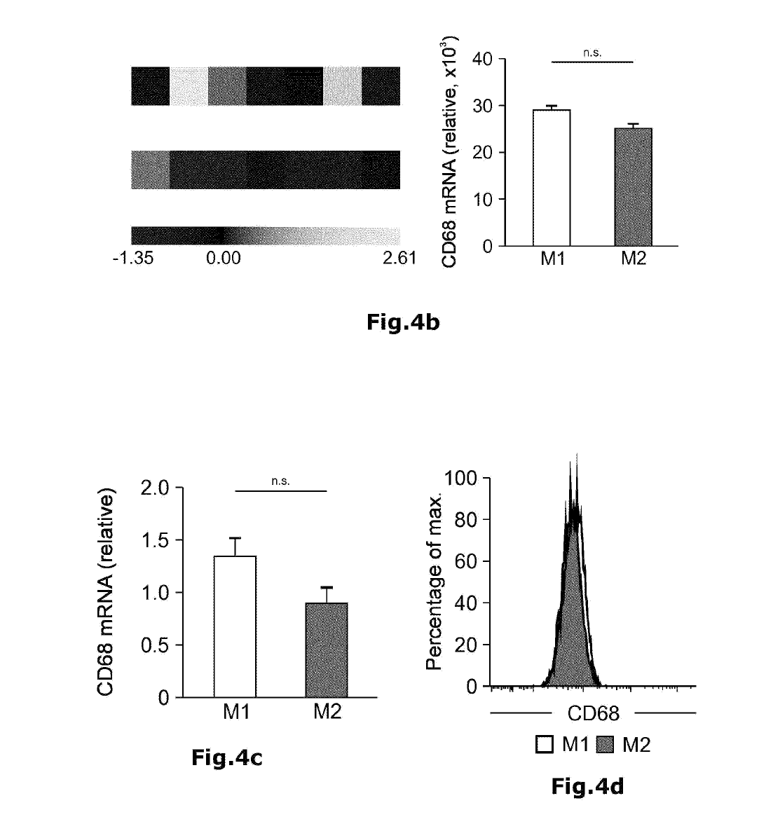

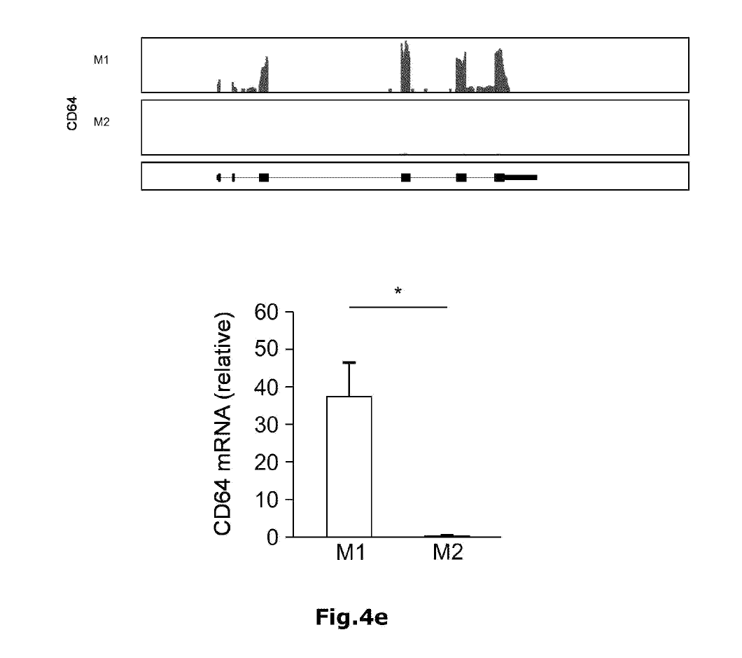

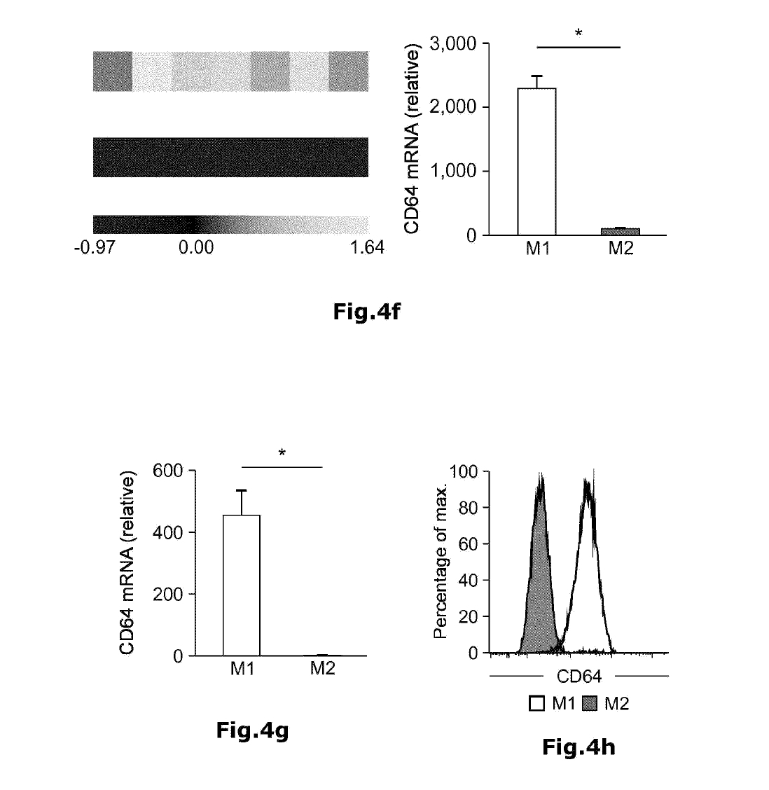

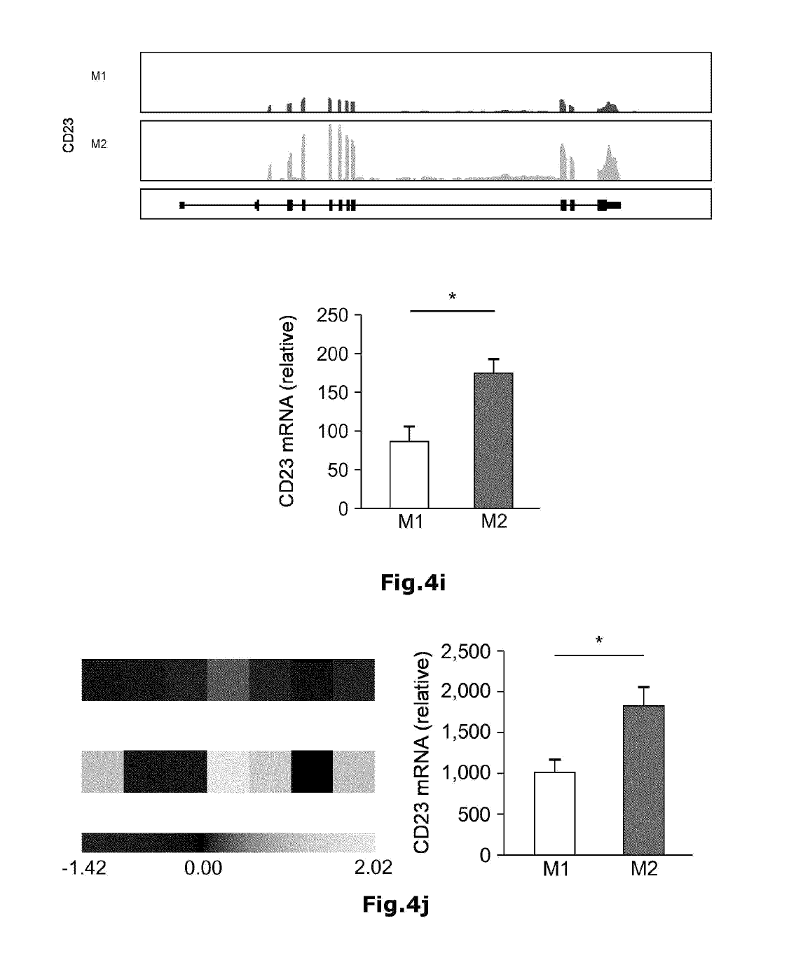

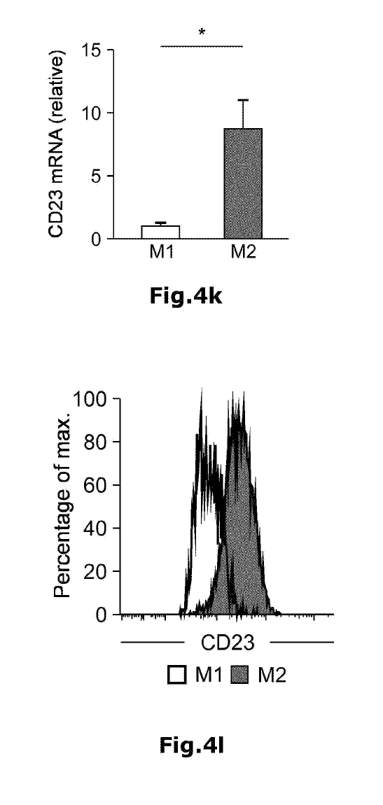

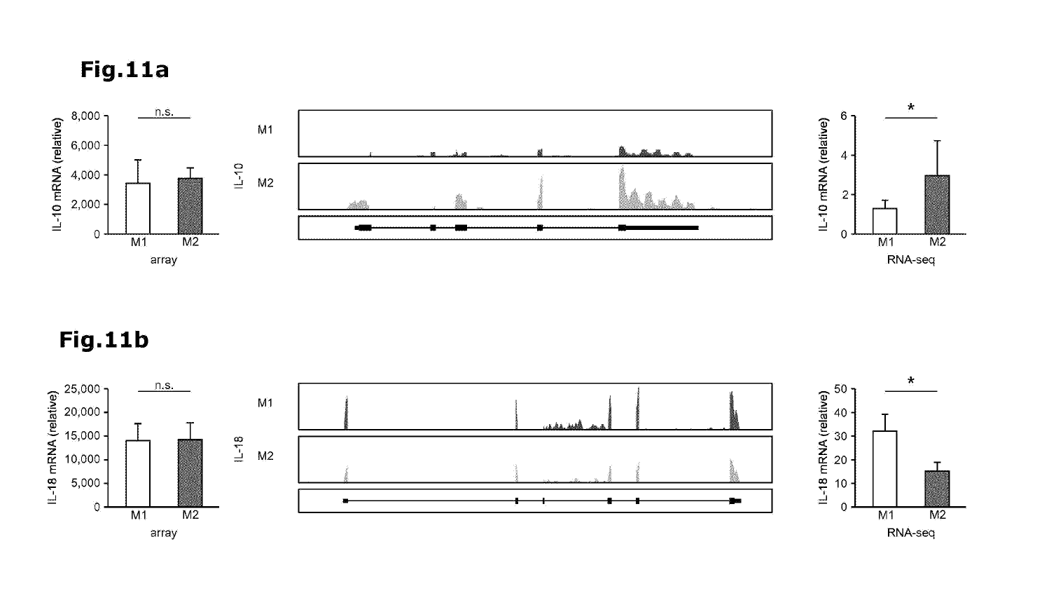

Exon resolution transcriptome analysis of known macrophage markers. Another advantage of RNA-seq is to resolve gene expression on the exon level (FIG. 4). For the macrophage related genes CD68 (FIG. 4a-d), CD64 (FIG. 4e-h) and CD23 (FIG. 4i-l), RNA-seq data were visualized for the genomic loci of the respective genes and compared with array, qPCR, and FACS data. For CD68, RNA-seq data revealed similarly high expression for M1 and M2 macrophages for all exons with little variance in expression levels between donors (FIGS. 4a and 11). Slightly higher variance was observed for both microarray (FIG. 4b) and qPCR data (FIG. 4c) while protein levels showed equal expression in all samples analyzed (FIG. 4d). For CD64, RNA-seq revealed complete absence of expression for all exons in M2-like macrophages with high expression in M1-like macrophages (FIG. 4e), which was similarly observed by other technologies (FIG. 4f-h). For CD23, protein data suggest significantly elevated expression on M2-like macrophages with low level expression on M1-like macrophages (FIG. 4l), a result which was also observed for RNA-seq data (FIG. 4i) as well as array (FIG. 4j) and qPCR (FIG. 4k). Similar results were obtained for other marker genes (data not shown). Additionally, we were able to detect classical M1/M2-markers not accessible using microarrays (FIG. 11), suggesting that high-resolution RNA-seq data are predestined for exploration of genes not detectable using microarrays, novel marker genes, as well as biological principles of macrophage polarization.

### Example 6

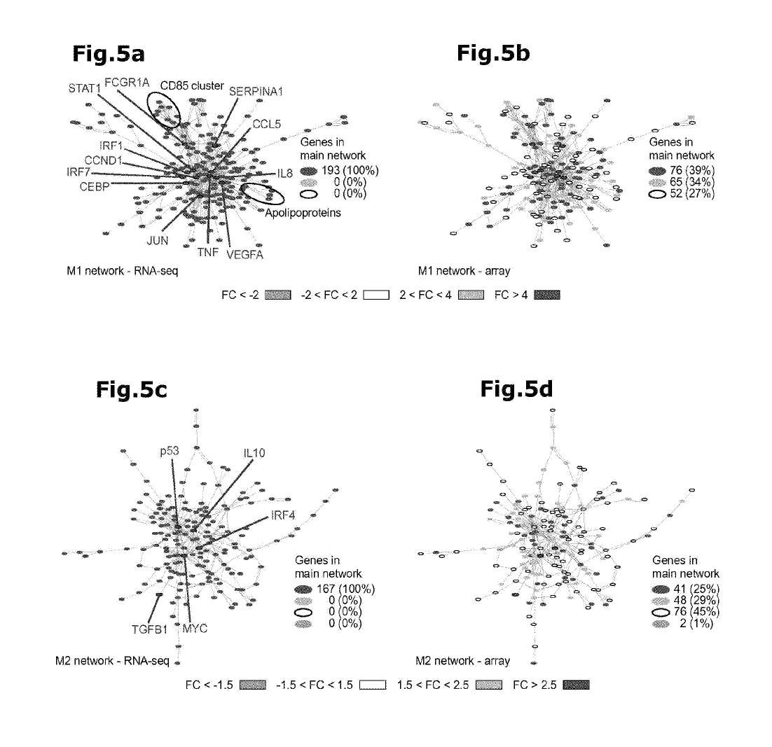

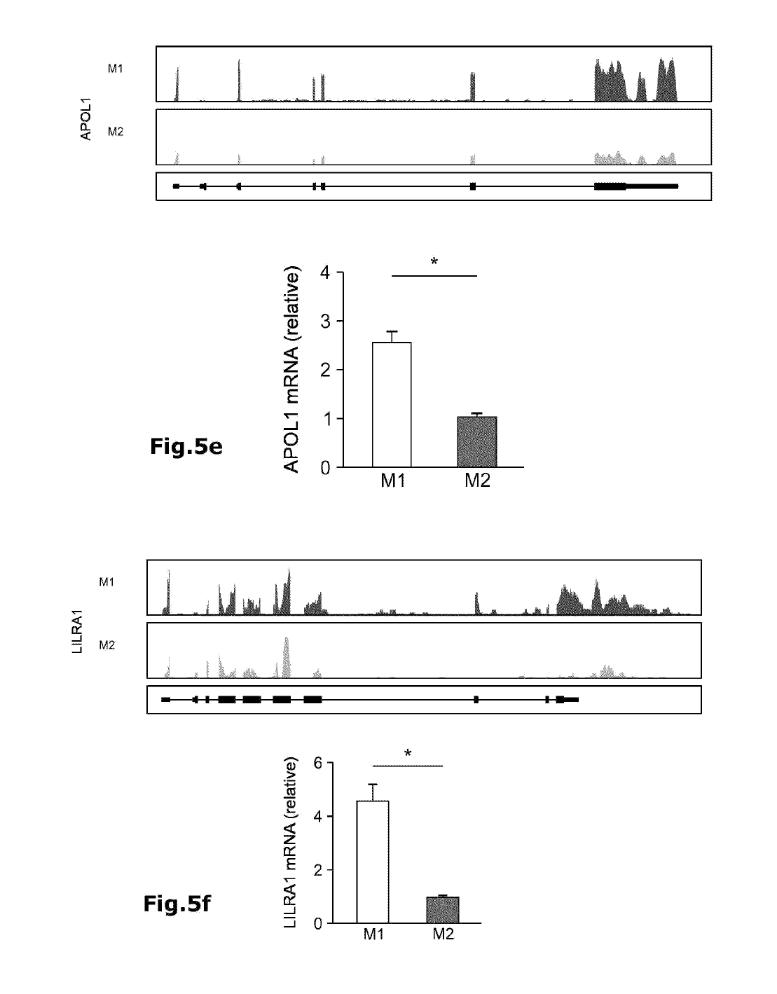

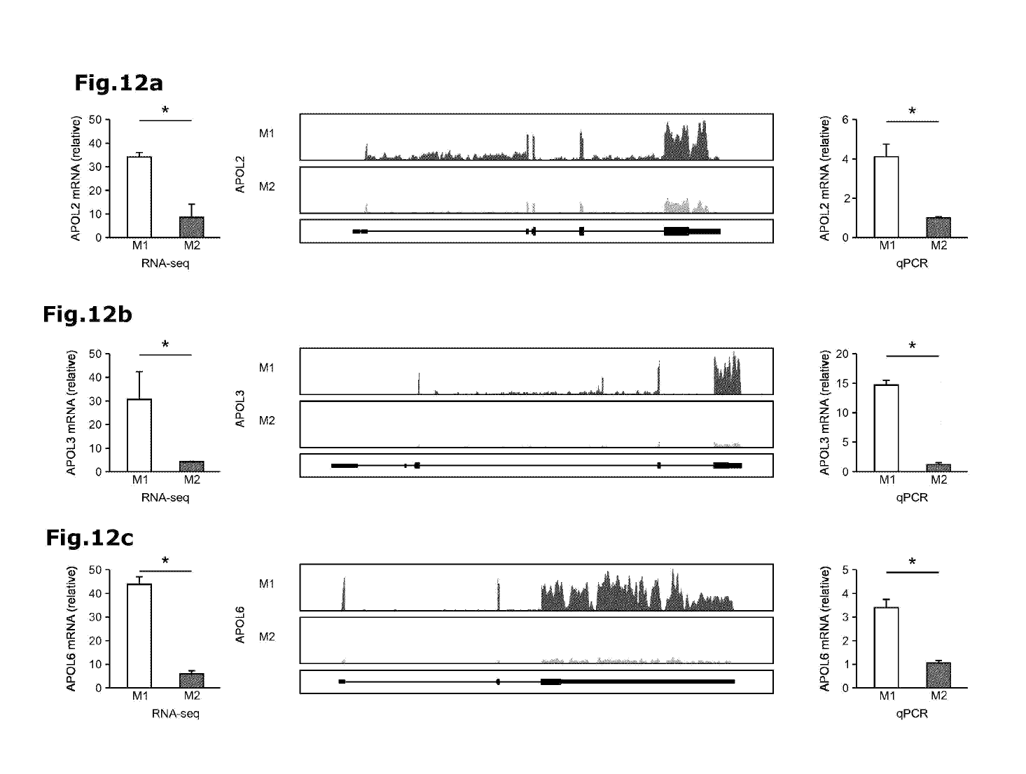

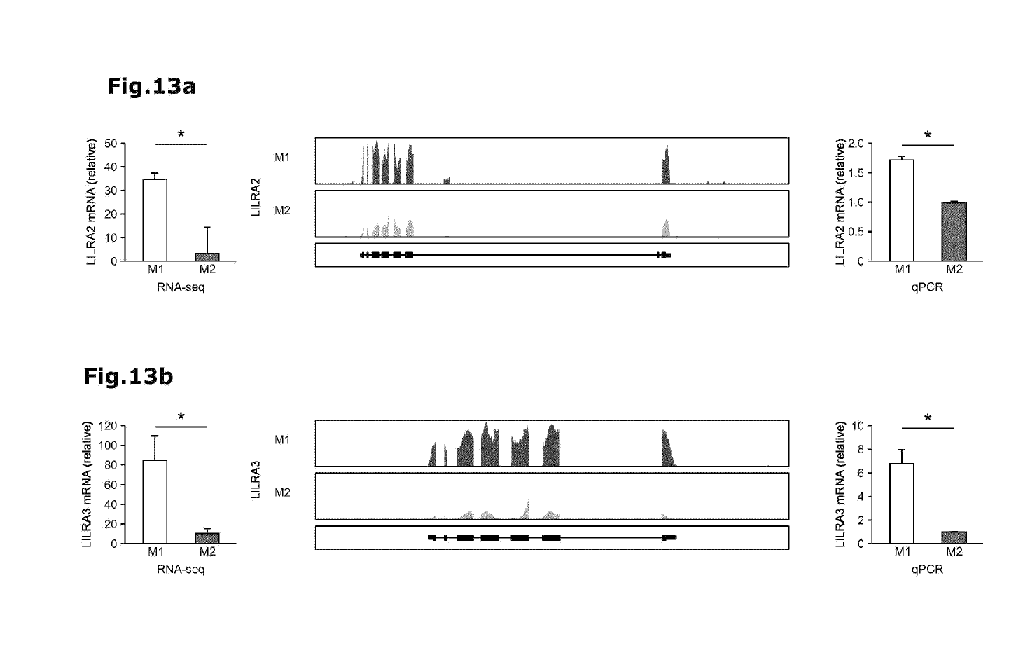

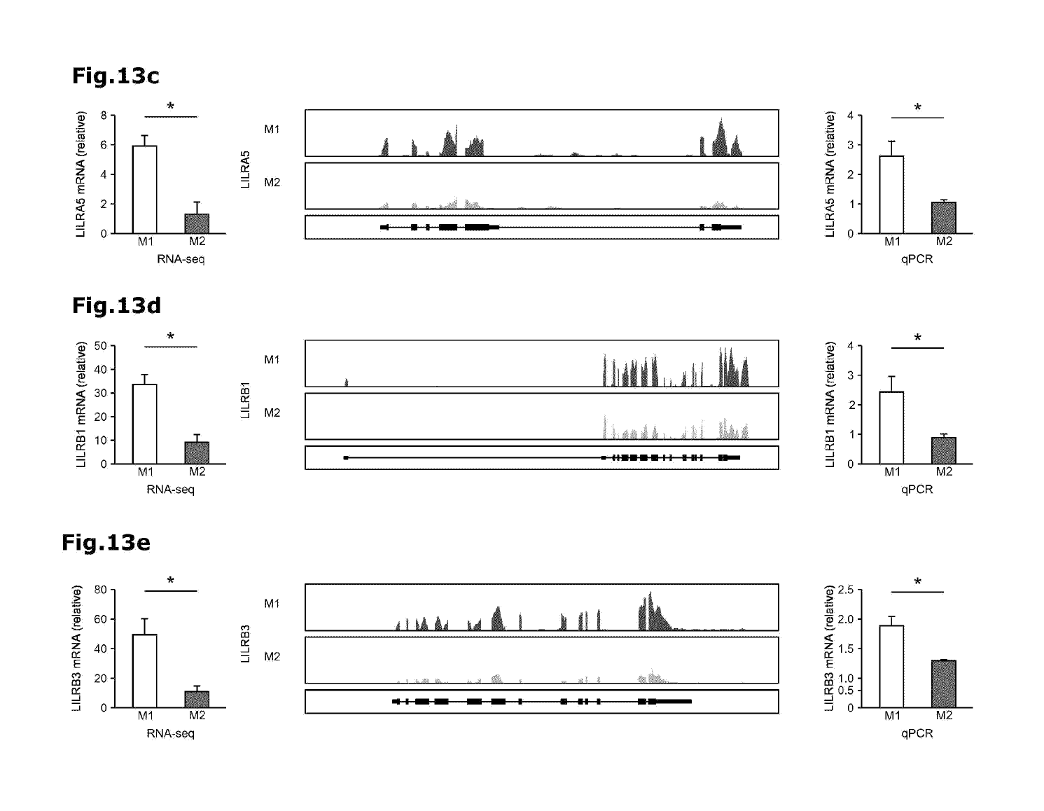

RNA-seq ameliorates network-based analysis in M1- and M2-like macrophages. To understand if RNA-seq would also enhance the understanding of biological principles of macrophage polarization we applied network analysis based on a priori information assessing the information content of RNA-seq data in comparison to array data. Genes expressed at elevated levels in M1 RNA-seq data (FC>4) were used for network generation (FIG. 5a). This primary RNA-seq based M1 network was subsequently used to visualize array-based gene expression (FIG. 5b). When genes at a lower level of differential expression (FC>2) were included 73% of the network was revealed in the array data and central hubs of the network were also categorized as being highly (FC>4) differentially expressed. However, only RNA-seq data revealed two gene clusters of immunomodulating proteins highly enriched in the M1 network, namely apolipoproteins L (APOL) (FIGS. 5a and 12) and the leukocyte immunoglobulin-like receptor (LILR) family (FIGS. 5a and 13) (Pays E., Vanhollebeke B., Curr Opin Immunol. 21(5):493-498 (2009); Samanovic M. et al., PLoS Pathog. 5(1):e1000276 (2009); Brown D. et al., Tissue Antigens. 64(3):215-225 (2004)). As exemplified for LILRA1 and APOL1 both genes were clearly identified by RNA-seq and qRT-PCR (FIGS. 5e and f) but not by microarray analysis (data not shown). Applying the RNA-seq data-based M2 network (FIG. 5c) to the array data (FIG. 5d) revealed only 54% elevated genes and major network hubs were not revealed at all. Taken together, RNA-seq data were clearly enriched for biological a priori information in both M1 and M2 polarization.

### Example 7

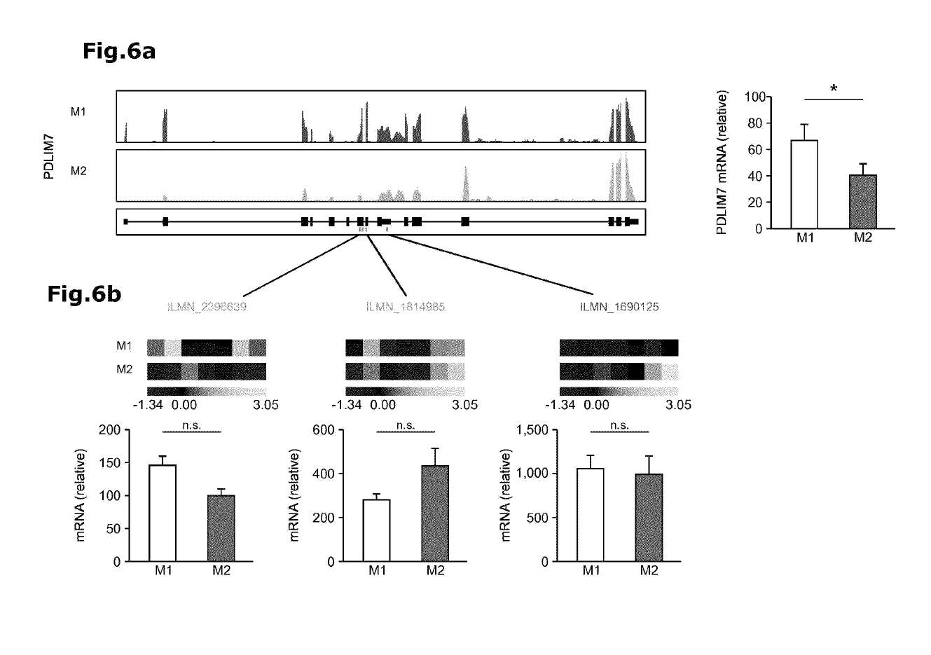

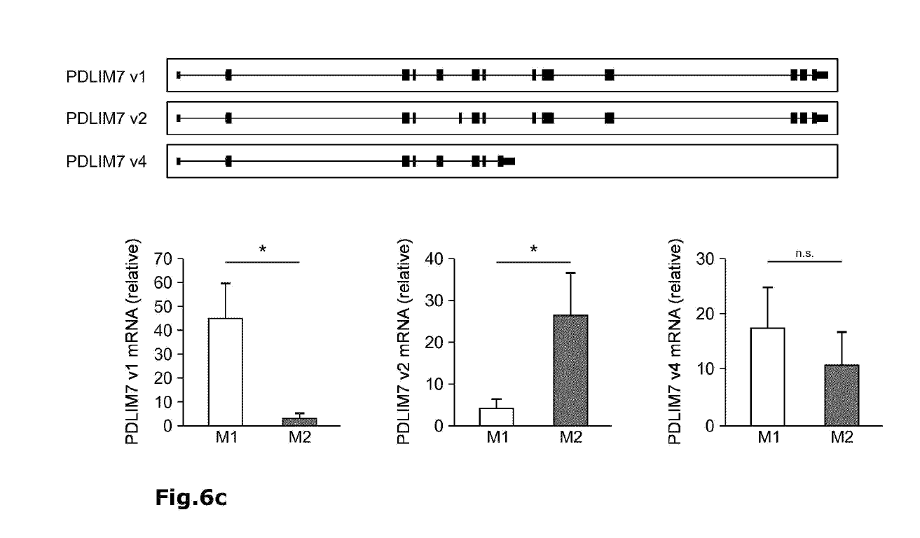

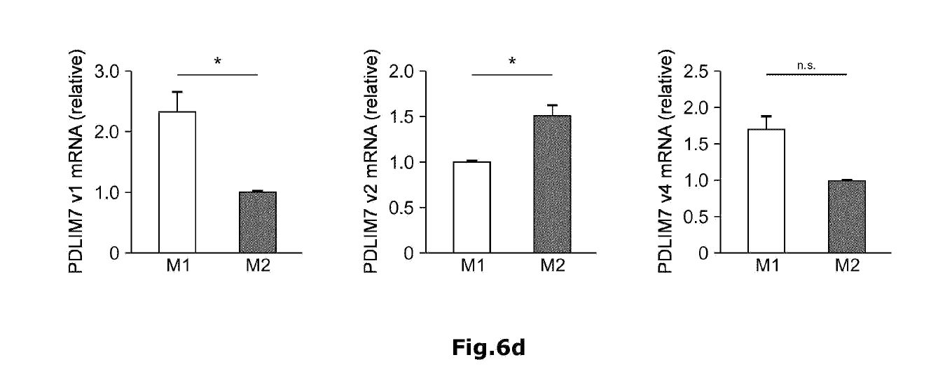

Identification of splice variants and RNA chimaera in differentially stimulated human macrophages. It has recently been suggested that cell differentiation can result in usage of alternative gene transcripts or isoform switching (Trapnell C. et al., Nature biotechnology 28(5):511-515 (2010)). We applied Cufflinks and Cuffdiff to illuminate switches in dominant promoter usage, transcription start sites (TSS), and coding sequences (CDS) (Trapnell C. et al., Nature biotechnology 28(5):511-515 (2010)). This analysis revealed 9 genes with alternative promoters (Table 4), 28 genes using alternative TSS (Table 5), and 20 genes with different CDS in M1- and M2-like macrophages (Table 6). We analyzed one of these genes in greater detail. For the gene encoding PDZ and LIM domain 7 (enigma) (PDLIM7) we observed a slight but significant increase in M1-like macrophages for the complete locus in RNA-seq data (FIG. 6a) while the probes on the microarray revealed no significant changes (FIG. 6b). Previous screening projects suggested three different CDS for PDLIM7. Applying Cufflinks and Cuffdiff to M1 and M2 RNA-seq data clearly revealed differential expression of individual CDS (FIG. 6c). While PDLIM7 v1 was mainly expressed by M1-like macrophages, M2-like macrophages mainly expressed PDLIM7 v2, while no difference was observed for PDLIM7 v4. We validated the usage of these variants by version-specific qPCR (FIG. 6d). Taken together, these new findings might open new avenues towards the role of alternative splicing in macrophages potentially linking alternative transcript usage with macrophage polarization.

### Example 8

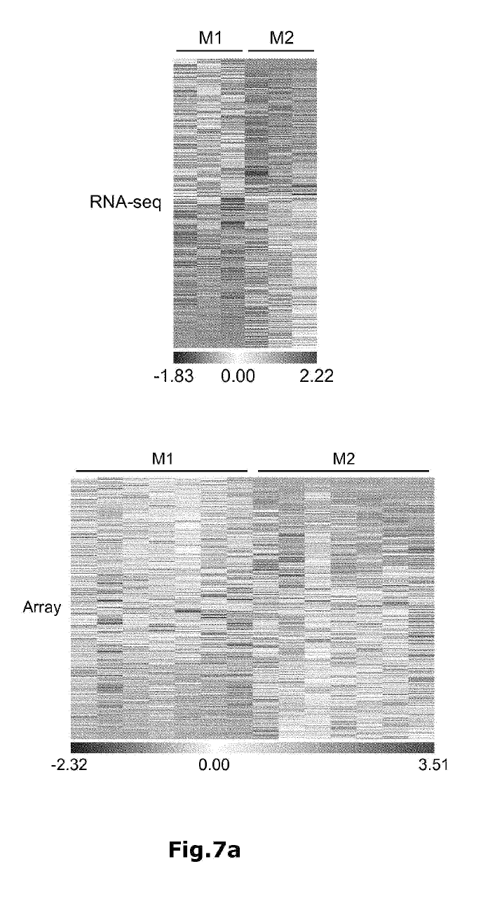

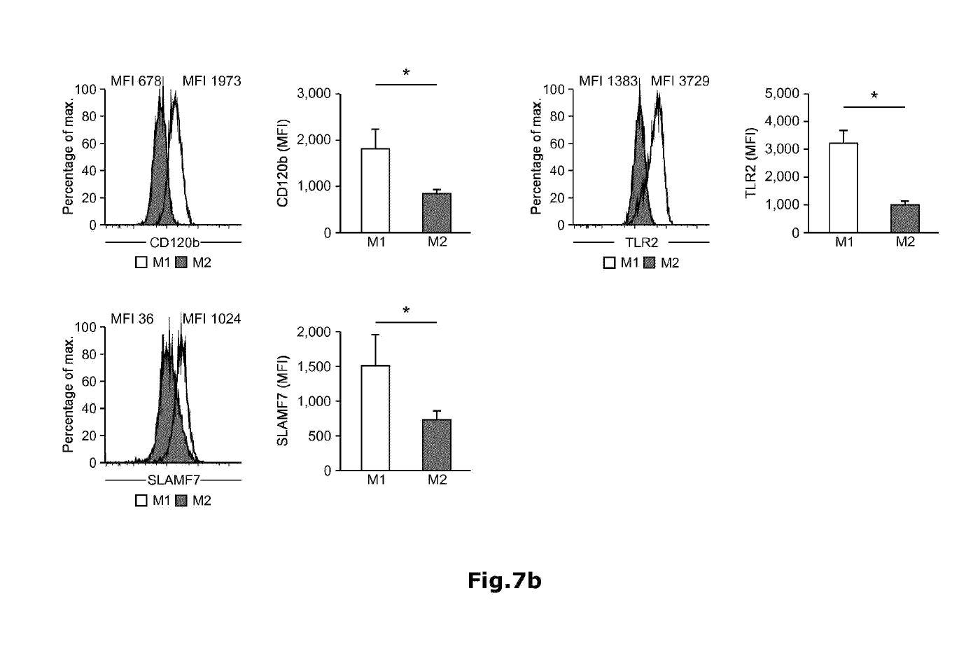

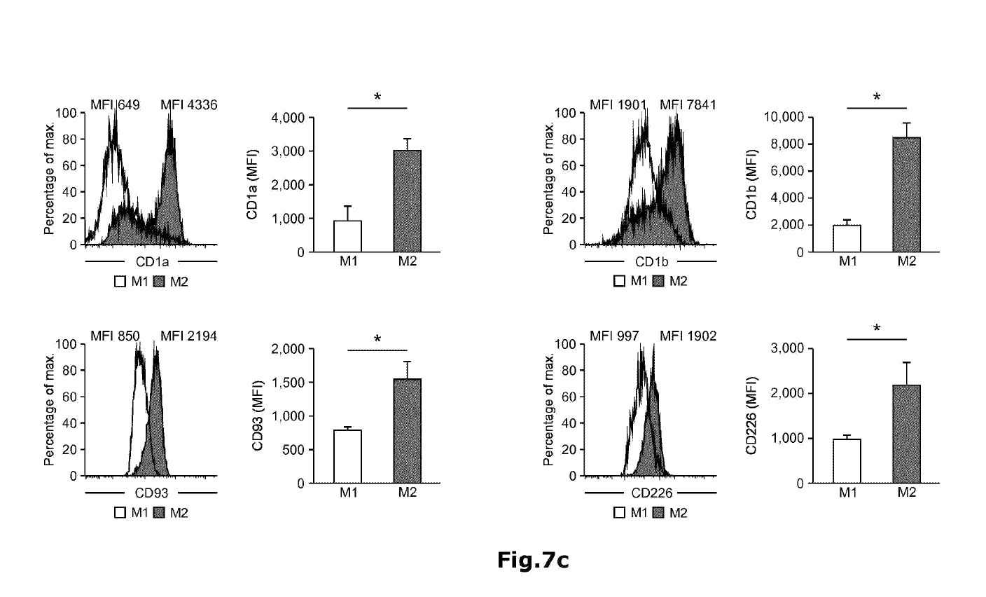

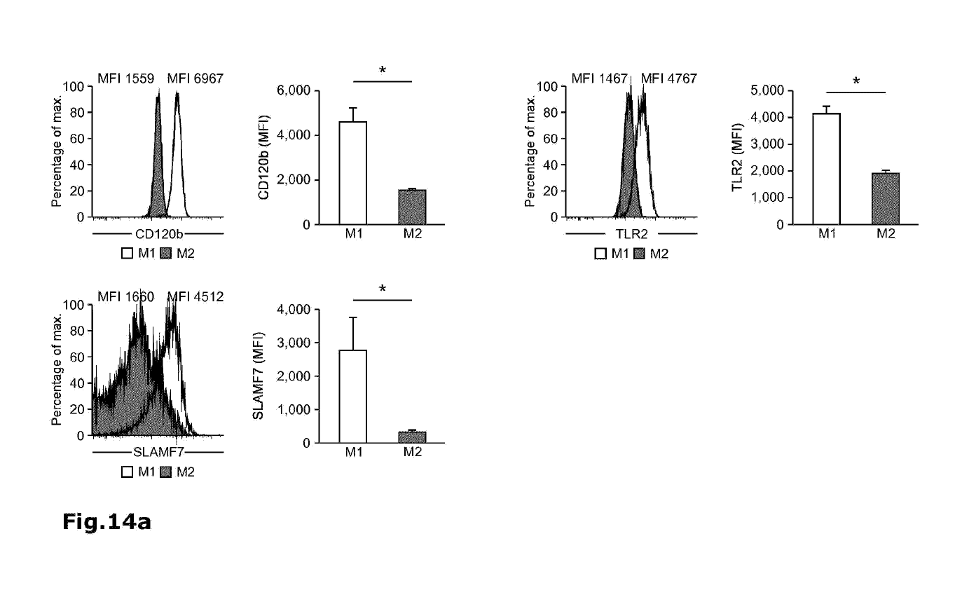

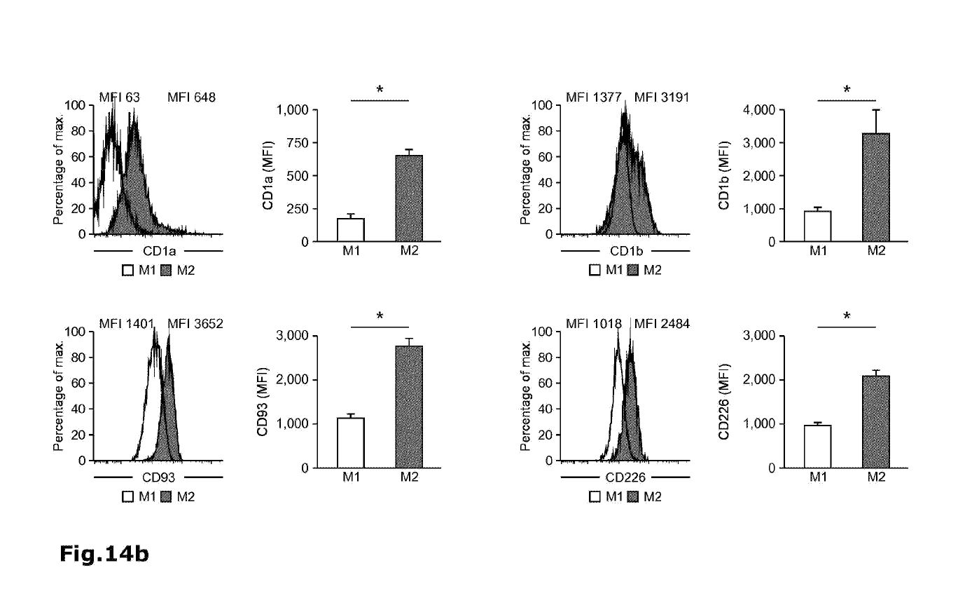

New markers for M1- and M2-like macrophages identified by combined transcriptome analysis. In light of the still limited number of cell surface markers clearly distinguishing human M1- from M2-like macrophages, we interrogated the genes of the human surfaceome (da Cunha J. P. et al., Proceedings of the National Academy of Sciences of the United States of America 106(39):16752-16757 (2009)) for differential expression between M1- and M2-like macrophages. By this approach 475 surface molecules were found to be differentially expressed (FIG. 7a). As visualized in FIG. 7b, the cell surface molecules CD120b, TLR2, and SLAMF7 showed preferential expression in M1-like macrophages, which was true irrespective of differentiation of macrophages by GM-CSF or M-CSF (FIG. 14a). Several surface molecules, including CD1a, CD1b, CD93 and CD226 were significantly increased in expression in M2-like macrophages (FIGS. 7c and 14b). Taken together, screening higher-resolution transcriptome data established by RNA-seq allows for the identification of novel genes related to specific polarization programs in macrophages.

### Tables

TABLE 1
RNA-Seq

| | M1 | | M2 | |
|---|---|---|---|---|
| | reads | percentage | reads | percentage |
| | (×10⁶) | (%) | (×10⁶) | (%) |
| Total | 15.0 ± 2.8 | | 20.4 ± 9.2 | |
| Exons | 11.8 ± 2.2 | 78.4 ± 1.1 | 16.1 ± 7.4 | 79.4 ± 2.0 |
| Exon-Intron | 0.4 ± 0.1 | 2.5 ± 0.1 | 0.5 ± 0.2 | 2.4 ± 0.2 |
| boundaries | | | | |
| Introns | 2.1 ± 0.5 | 14.1 ± 1.0 | 2.7 ± 1.3 | 13.2 ± 1.6 |
| Intergenic | 0.8 ± 0.1 | 5.0 ± 0.2 | 1.0 ± 0.5 | 5.0 ± 0.3 |
| regions | | | | |

TABLE 2
qPCR oligonucleotides

| | | |
|---|---|---|
| CD68 Forward | 5′-GTCCACCTCGACCTGCTCT-3′ | (SEQ ID NO: 15) |
| CD68 Reverse | 5′-CACTGGGGCAGGAGAAACT-3′ | (SEQ ID NO: 16) |
| CD64 Forward | 5′-TGGGAAAGCATCGCTACAC-3′ | (SEQ ID NO: 17) |
| CD64 Reverse | 5′-GCACTGGAGCTGGAAATAGC-3′ | (SEQ ID NO: 18) |
| CD23 Forward | 5′-GGGAGAATCCAAGCAGGAC-3′ | (SEQ ID NO: 19) |
| CD23 Reverse | 5′-GGAAGCTCCTCGATCTCTGA-3′ | (SEQ ID NO: 20) |
| LILRA1 Forward | 5′-TCGCTGTTTCTACGGTAGCC-3′ | (SEQ ID NO: 21) |
| LILRA1 Reverse | 5′-GGGTGGGTTTGATGTAGGC-3′ | (SEQ ID NO: 22) |
| LILRA2 Forward | 5′-CAGCCACAATCACTCATCAGA-3′ | (SEQ ID NO: 23) |
| LILRA2 Reverse | 5′-AGGGTGGGTTTGCTGTAGG-3′ | (SEQ ID NO: 24) |
| LILRA3 Forward | 5′-GGAGCTCGTGGTCTCAGG-3′ | (SEQ ID NO: 25) |
| LILRA3 Reverse | 5′-CTTGGAGTCGGACTTGTTTTG-3′ | (SEQ ID NO: 26) |
| LILRA5 Forward | 5′-GCACCATGGTCTCATCCAT-3′ | (SEQ ID NO: 27) |
| LILRA5 Reverse | 5′-GTGGCTTTGGAGAGGTTCC-3′ | (SEQ ID NO: 28) |
| LILRB1 Forward | 5′-GGAGCTCGTGGTCTCAGG-3′ | (SEQ ID NO: 29) |
| LILRB1 Reverse | 5′-GGGTTTGATGTAGGCTCCTG-3′ | (SEQ ID NO: 30) |
| LILRB3 Forward | 5′-GGAGATACCGCTGCCACTAT-3′ | (SEQ ID NO: 31) |
| LILRB3 Reverse | 5′-GGTGGGTTTGCTGTAGGC-3′ | (SEQ ID NO: 32) |
| APOL1 Forward | 5′-TTCGAATTCCTCGGTATATCTTG-3′ | (SEQ ID NO: 33) |
| APOL1 Reverse | 5′-CACCTCCAGTTATGCGTCTG-3′ | (SEQ ID NO: 34) |
| APOL2 Forward | 5′-ATGATGAAGCCTGGAATGGA-3′ | (SEQ ID NO: 35) |
| APOL2 Reverse | 5′-TCAGAGCTTTACGGAGCTCAT-3′ | (SEQ ID NO: 36) |
| APOL3 Forward | 5′-GCCTGGAAGAGATTCGTGAC-3′ | (SEQ ID NO: 37) |
| APOL3 Reverse | 5′-CTTCAGAGCTTCGTAGAGAGCA-3′ | (SEQ ID NO: 38) |
| APOL6 Forward | 5′-AAGTGAGGCTGGTGTTGGTT-3′ | (SEQ ID NO: 39) |
| APOL6 Reverse | 5′-CGTCTTGTAGCTCCACGTCTT-3′ | (SEQ ID NO: 40) |
| GAPDH Forward | 5′-AGCCACATCGCTCAGACAC-3′ | (SEQ ID NO: 41) |
| GAPDH Reverse | 5′-GCCCAATACGACCAAATCC-3′ | (SEQ ID NO: 42) |

TABLE 3
Isoform specific qPCR oligonucleotides

| | | |
|---|---|---|
| PDLIM7 v1 Forward | 5′-CCGCAGCAGAATGGACAG-3′ | (SEQ ID NO: 43) |
| PDLIM7 v1 Reverse | 5′-GCTCCTGGGGTGTAGATG-3′ | (SEQ ID NO: 44) |
| PDLIM7 v2 Forward | 5′-ACCGCAGAAGGTGCAGAC-3′ | (SEQ ID NO: 45) |
| PDLIM7 v2 Reverse | 5′-CTGGCTTGATTTCTTCAGGTG-3′ | (SEQ ID NO: 46) |
| PDLIM7 v4 Forward | 5′-CCGCAGCAGAATGGACAG-3′ | (SEQ ID NO: 47) |
| PDLIM7 v4 Reverse | 5′-GCAGGAGGAACAGAAAGAG-3′ | (SEQ ID NO: 48) |
| GAPDH Forward | 5′-AGCCACATCGCTCAGACAC-3′ | (SEQ ID NO: 49) |
| GAPDH Reverse | 5′-GCCCAATACGACCAAATCC-3′ | (SEQ ID NO: 50) |

TABLE 4
Alternative promoter usage

| | |
|---|---|
| C8orf83 | chr8: 93895757-93978372 |
| EIF4ENIF1 | chr22: 31835344-31885874 |
| HRH1 | chr3: 11178778-11304939 |
| JDP2 | chr14: 75894508-75939404 |
| NCS1 | chr9: 132934856-132999583 |
| PDE2A | chr11: 72287184-72385494 |
| PUS7L | chr12: 44122409-44152596 |
| SMARCD3 | chr7: 150936058-150974231 |
| TSC22D1 | chr13: 45007654-45154568 |

TABLE 5
Alternative TSS usage

| | |
|---|---|
| WFS1 | chr4: 6271576-6304992 |
| ARNT | chr1: 150768686-150849186 |
| ASRGL1 | chr11: 62104773-62160887 |
| OSBPL9 | chr1: 52082546-52344609 |
| GBA | chr1: 155204238-155214653 |
| HES6 | chr2: 239146907-239148681 |
| BAT5 | chr6: 31654725-31671137 |
| DCTN1 | chr2: 74588280-74621008 |
| RASGRP4 | chr19: 38893774-38916945 |
| SNX16 | chr8: 82711817-82754521 |
| NDUFC1 | chr4: 140211070-140311935 |
| CCDC17 | chr1: 46085715-46089731 |
| CD200R1 | chr3: 112641531-112693937 |
| FECH | chr18: 55212072-55253969 |
| NRGN | chr11: 124609828-124617102 |
| RB1CC1 | chr8: 53535017-53627026 |
| UBQLN1 | chr9: 86274877-86323168 |
| MTERFD3 | chr12: 107371068-107380929 |
| MBOAT7 | chr19: 54677105-54693733 |
| RANBP3 | chr19: 5914192-5978320 |
| RAP1GDS1 | chr4: 99182526-99365012 |
| TNNT1 | chr19: 55644160-55660606 |
| ABCC1 | chr16: 16043433-16236931 |
| CDCA7L | chr7: 21582832-21985542 |
| HYI | chr1: 43888796-43919660 |
| C8orf83 | chr8: 93895757-93978372 |
| CD36 | chr7: 80231503-80308593 |
| NT5C3 | chr7: 33053741-33102409 |

TABLE 6
Alternative CDS usage

| | |
|---|---|
| ABCC1 | chr16: 16043433-16236931 |
| CCDC17 | chr1: 46085715-46089731 |
| CD200R1 | chr3: 112641531-112693937 |
| CDCA7L | chr7: 21582832-21985542 |
| FECH | chr18: 55212072-55253969 |
| HES6 | chr2: 239146907-239148681 |
| HYI | chr1: 43888796-43919660 |
| JDP2 | chr14: 75894508-75939404 |
| MYO1B | chr2: 192110106-192290115 |
| NCS1 | chr9: 132934856-132999583 |
| PDLIM7 | chr5: 176910394-176924602 |
| RANBP3 | chr19: 5914192-5978320 |
| RAP1GDS1 | chr4: 99182526-99365012 |
| RASGRP4 | chr19: 38893774-38916945 |
| RB1CC1 | chr8: 53535017-53627026 |
| RP6-213H19.1 | chrX: 131157244-131209971 |
| SLC25A45 | chr11: 65142662-65150142 |
| SNX16 | chr8: 82711817-82754521 |
| TNNT1 | chr19: 55644160-55660606 |
| UBQLN1 | chr9: 86274877-86323168 |

### Sequence Listing, Free Text

SEQ ID NO: Description
1/2 tumor necrosis factor receptor superfamily, member 1B (CD120b)
3/4 toll-like receptor 2 (TLR2)
5/6 SLAM family member 7 (SLAMF7)
7/8 CD1a
9/10 CD1b
11/12 CD93
14/15 CD226
16-50 primer

1. A method for identifying of, distinguishing between and isolating of M1-like and M2-like macrophages which comprises characterizing the macrophages based on the relative abundance of one or more of the specific M1-associated cell surface markers CD120b, TLR2 and SLAMF7, or of one or more of the specific M2-associated cell surface markers CD1a, CD1b, CD93 and CD226, respectively.

2. The method of claim 1, wherein the relative abundance of the M1-associated cell surface markers is higher in M1-like macrophages than in M2-like macrophages and the relative abundance of the M2-associated cell surface markers is higher in M2-like macrophages than in M1-like macrophages.

3. The method of claim 1, wherein the identifying of and distinguishing between the M1-like and M2-like macrophages is performed by an amplification or by a targeted resequencing of one or more of the specific M1-associated cell surface marker nucleic acids CD120b, TLR2 and SLAMF7 (SEQ ID NOs: 1, 3 and 5), or of one or more of the specific M2-associated cell surface marker nucleic acids CD1a, CD1b, CD93 and CD226 (SEQ ID NOs: 7, 9, 11 and 13), respectively, of the macrophage, and a subsequent detection of the amplification/resequencing product.

4. The method of claim 3, wherein the amplification/resequencing employs one or more primers derived from each of the marker genes.

5. The method of claim 1, wherein the identifying of and distinguishing between the M1-like and M2-like macrophages comprises hybridizing one or more probes selective for one of the specific M1-associated cell surface marker nucleic acids CD120b, TLR2 and SLAMF7 (SEQ ID NOs: 1, 3 and 5), or for one of the specific M2-associated cell surface marker nucleic acids CD1a, CD1b, CD93 and CD226 (SEQ ID NOs: 7, 9, 11 and 13), respectively, of the macrophage.

6. The method of claim 5, which is performed on a hybridization array.

7. The method of claim 1, wherein the identifying of, distinguishing between and isolating of the M1-like and M2-like macrophages comprises contacting the macrophages with one or more binding molecules directed against the specific M1-associated cell surface marker protein CD120b, TLR2 and SLAMF7 (SEQ ID NOs: 2, 4 and 6), or with one or more binding molecules directed against the specific M2-associated cell surface marker nucleic acids CD1a, CD1b, CD93 and CD226 (SEQ ID NOs: 8, 10, 12 and 14), respectively.

8. The method of claim 7, wherein the binding molecules
(i) are antibodies; and/or
(ii) are labeled with maker molecules.

9. The method of claim 8, wherein the binding molecules are selected from FITC-labeled CD1b, CD93, CD226 and anti-TLR2; PE-labeled CD120b and anti-SLAMF7; PE-Cy5-labeled CD1a monoclonal antibodies.

10. The method according to claim 7, which is performed on a FACS sorter.

11. A kit for performing the method according to claim 1, which comprises at least one reagent for identifying of, distinguishing between and isolating of M1-like macrophages and at least one reagent for identifying of, distinguishing between and isolating of M2-like macrophages, said reagents being selected from
(i) one or more primers derived from one or more of the specific M1-associated cell surface marker nucleic acids CD120b, TLR2 and SLAMF7 (SEQ ID NOs: 1, 3 and 5), or of one or more of the specific M2-associated cell surface marker nucleic acids CD1a, CD1b, CD93 and CD226 (SEQ ID NOs: 7, 9, 11 and 13), respectively, of the macrophage,
(ii) one or more probes selective for one of the specific M1-associated cell surface marker nucleic acids CD120b, TLR2 and SLAMF7 (SEQ ID NOs: 1, 3 and 5), or for one of the specific M2-associated cell surface marker nucleic acids CD1a, CD1b, CD93 and CD226 (SEQ ID NOs: 7, 9, 11 and 13), respectively, of the macrophage,
(iii) a hybridization array, or
(iv) one or more binding molecules directed against the specific M1-associated cell surface marker protein CD120b, TLR2 and SLAMF7 (SEQ ID NOs: 2, 4 and 6), or directed against the specific M2-associated cell surface marker nucleic acids CD1a, CD1b, CD93 and CD226 (SEQ ID NOs: 8, 10, 12 and 14), respectively.

12. Method of using antibodies selected from CD120b, TLR2 and SLAMF7 antibodies for identifying, characterizing and isolating M1-like macrophages.

13. Method of using antibodies selected from CD1a, CD1b, CD93 and CD226 antibodies for identifying, characterizing and isolating M2-like macrophages.

14. A population of M1-like macrophages isolated by the method of claim 7.

15. A population of M2-like macrophages isolated by the method of claim 7.
